SEQUENCE LISTING (8 sequences)

SEQ ID NO: 1 - RNA, 22 nt, synthetic construct
  source 1..22: mol_type = other RNA
tattttctgcattcgcccttgc

SEQ ID NO: 2 - RNA, 24 nt, synthetic construct
  source 1..24: mol_type = other RNA
aaggagcgatttggagaaaataaa

SEQ ID NO: 3 - DNA, 52 bp, synthetic construct
  source 1..52: mol_type = other DNA
cagaaacataacatcagagttgttacagtgagtaacaatatggttgaagggc

SEQ ID NO: 4 - DNA, 30 bp, synthetic construct
  source 1..30: mol_type = other DNA
  modified_base 30: mod_base = OTHER; note = Locked nucleic acid
  misc_feature 1..27: note = DNA
  misc_feature 28..30: note = RNA
aagcagtggtatcaacgcagagtacatggg

SEQ ID NO: 5 - DNA, 23 bp, synthetic construct
  source 1..23: mol_type = other DNA
ggacagaaacataacatcagagt

SEQ ID NO: 6 - DNA, 17 bp, synthetic construct
  source 1..17: mol_type = other DNA
cagtggtatcaacgcag

SEQ ID NO: 7 - DNA, 26 bp, synthetic construct
  source 1..26: mol_type = other DNA
tggttgaagggcgaatgcagaaaata

SEQ ID NO: 8 - DNA, 33 bp, synthetic construct
  source 1..33: mol_type = other DNA
  modified_base 1: mod_base = OTHER; note = Isocytosine
  modified_base 2: mod_base = OTHER; note = Isoguanosine
  modified_base 3: mod_base = OTHER; note = Isocytosine
  modified_base 33: mod_base = OTHER; note = Locked nucleic acid
  misc_feature 1..30: note = DNA
  misc_feature 31..33: note = RNA
cgcaagcagtggtatcaacgcagagtacatggg

(19) United States
(12) Patent Application Publication
(10) Pub. No.: US 2025/0163504 A1
(43) Pub. Date: May 22, 2025

(54) PROCESSING METHOD FOR SMALL RNAS

(71) Applicant: TATAA Biocenter AB, Göteborg (SE)

(72) Inventors: Robert SJÖBACK, Partille (SE); Anna ÅSMAN, Surte (SE)

(21) Appl. No.: 18/950,504

(22) Filed: Nov. 18, 2024

Related U.S. Application Data
(60) Provisional application No. 63/601,168, filed on Nov. 20, 2023.

(51) Int. Cl. C12Q 1/6851; C12Q 1/6876

(52) U.S. Cl. CPC C12Q 1/6851; C12Q 1/6876; C12Q 2600/178

(57) ABSTRACT

Methods, compositions, and systems for processing small RNAs are described herein.

### CROSS-REFERENCE

This application claims the benefit of U.S. Provisional Application No. 63/601,168, filed on Nov. 20, 2023, which is incorporated by reference herein in its entirety.

### SEQUENCE LISTING

The instant application contains a Sequence Listing which has been submitted electronically in XML file format and is hereby incorporated by reference in its entirety. Said XML copy, created on Nov. 13, 2024, is named 46005-703_201_SL.xml and is 12,080 bytes in size.

### BACKGROUND

Small RNA molecules have emerged as critical regulators in the expression and function of eukaryotic genomes. Two primary categories of small RNAs-short interfering RNAs (siRNAs) and microRNAs (miRNAs)—can act in both somatic and germline line-ages in a broad range of eukaryotic species to regulate endogenous genes and to defend the genome from invasive nucleic acids.

### SUMMARY

In some aspects, the present disclosure provides for a method for processing a ribonucleic acid (RNA) molecule, comprising: (a) providing a reaction mixture configured to produce a complementary deoxyribonucleic acid (cDNA) molecule from the RNA molecule, wherein the reaction mixture comprises: (i) the RNA molecule; (ii) a stem-loop primer that comprises: a 5′ hemiprobe segment configured to hybridize to a first end region of the RNA molecule; a stem-loop segment; and a 3′ hemiprobe segment configured to hybridize to a second end region of the RNA molecule, and (iii) a template switch oligonucleotide (TSO) that provides an exogenous template, wherein the TSO is configured for elongation of the RNA molecule when the RNA molecule is reverse-transcribed. In some embodiments, the exogenous template comprises a sequence exogenous to the RNA molecule. In some embodiments, the exogenous template comprises fewer than or equal to 10 consecutive bases identical to the RNA molecule. In some embodiments, a complement of the exogenous template is not configured to bind to the RNA molecule. In some embodiments, the RNA molecule is a naturally-occurring RNA molecule. In some embodiments, the RNA molecule is obtained from a cell, a tissue, or an organism. In some embodiments, the exogenous template comprises a sequence exogenous to the cell, the tissue, or the organism. In some embodiments, the reaction mixture further comprises a cell or cellular RNA. In some embodiments, the cell or cellular RNA comprises coding or noncoding RNA at least partially complementary to the RNA molecule. In some embodiments, the method further comprises producing the cDNA molecule from the RNA molecule. In some embodiments, the stem-loop segment comprises a first synthetic primer hybridization sequence. In some embodiments, the exogenous template of the TSO comprises a reverse complement of a second synthetic primer hybridization sequence. In some embodiments, the method further comprises detecting the cDNA molecule via the first synthetic primer hybridization sequence and the second synthetic primer hybridization sequence. In some embodiments, the detecting further comprises quantitative PCR (qPCR), digital PCR (dPCR), sequencing, or array hybridization. In some embodiments, the detecting further comprises contacting the cDNA molecule with a probe oligo configured to hybridize to an internal region of the cDNA molecule. In some embodiments, the probe oligo is a hybridization probe or a hydrolysis probe. In some embodiments, the internal region of the cDNA molecule comprises a sequence complementary to the RNA molecule. In some embodiments, the internal region of the cDNA molecule comprises the stem-loop segment within the cDNA molecule. In some embodiments, the detecting the cDNA molecule further comprises combining in the reaction mixture or a second reaction mixture under conditions suitable to extend the cDNA molecule: (i) the cDNA molecule; (ii) a forward primer configured to hybridize to the first synthetic primer hybridization sequence; and (iii) a reverse primer. In some embodiments, the reverse primer is configured to hybridize to the second synthetic primer hybridization sequence. In some embodiments, wherein the TSO comprises a 3′ end region comprising rGrG. In some embodiments, the TSO comprises a 3′ terminal locked nucleic acid guanine. In some embodiments, the TSO comprises a 3′ end region comprising rGrG+G, wherein rG denotes riboguanosine and +G denotes locked nucleic acid (LNA) guanosine. In some embodiments, the TSO comprises a 3′ end region comprising at least one isoguanine (iG) or isocytosine (iC) residue. In some embodiments, the 3′ end region comprising at least one isoguanine or isocytosine residue comprises iGiC. In some embodiments, the stem-loop segment is non-overlapping with the 5′ hemiprobe segment and the 3′ hemiprobe segment. In some embodiments, the RNA molecule is fewer than 30 nucleotides in length. In some embodiments, the RNA molecule is a short interfering RNA (siRNA) or a microRNA (miRNA). In some embodiments, the RNA molecule comprises a synthetic sugar-modified oligonucleotide or a synthetic linkage between two nucleotides of the RNA molecule. In some embodiments, the RNA molecule comprises a phosphorothioate linkage, a 2′-fluororibonucleotide, a 2′-O-methylribonucleotide, or a 2′-O-methoxyethylribonucleotide. In some embodiments, the reaction mixture comprises a reverse transcriptase. In some embodiments, the reverse transcriptase comprises Maxima™ H Minus reverse transcriptase, SuperScript™ II reverse transcriptase, Superscript™ III reverse transcriptase, SuperScript™ IV reverse transcriptase, Template Switching RT enzyme mix, SMARTscribe reverse transcriptase, PrimeScript™ RT, or any combination thereof. In some embodiments, the 5′ hemiprobe is about 5 to about 20 nucleotides in length. In some embodiments, the 3′ hemiprobe is about 3 to about 10 nucleotides in length. In some embodiments, the stem-loop segment is about 20 to about 80 nucleotides in length. In some embodiments, the first synthetic primer hybridization sequence comprises a sequence according to 5′-GGACAGAAACATAACATCAGAGT-3′ (SEQ ID NO: 5) or a sequence having at least 90% sequence identity thereto. In some embodiments, the second synthetic primer hybridization sequence comprises a sequence according to 5′-CAGTGGTATCAACGCAG-3′ (SEQ ID NO: 6) or a sequence having at least 90% sequence identity thereto.

In some aspects, the present disclosure provides for a method for processing an RNA molecule, comprising: (a) providing in a reaction mixture under conditions suitable for extending the RNA molecule: (i) the RNA molecule, which comprises a synthetic oligonucleotide or a synthetic linkage between two nucleotides of the RNA molecule; and (ii) a stem-loop primer that comprises: a 5′ hemiprobe segment configured to hybridize to a first end region of the RNA molecule; a stem-loop segment comprising a first synthetic primer hybridization sequence; and a 3′ hemiprobe segment configured to hybridize to a second end region of the RNA molecule; and (b) extending the stem-loop primer using the RNA molecule as a template under conditions suitable to incorporate a 3′ region comprising a second synthetic primer hybridization sequence, thereby generating a cDNA molecule from the RNA molecule and the stem-loop primer. In some embodiments, the method further comprises detecting the cDNA molecule via the first synthetic primer hybridization sequence and the second synthetic primer hybridization sequence. In some embodiments, a complement of the second synthetic primer hybridization sequence is not configured to bind to the RNA molecule. In some embodiments, the RNA molecule is obtained from a cell, a tissue, or an organism. In some embodiments, the second synthetic primer hybridization sequence is exogenous to the cell, the tissue, or the organism. In some embodiments, the reaction mixture further comprises a cell or cellular RNA. In some embodiments, the cell or cellular RNA comprises RNA molecules at least partially complementary to the RNA molecule. In some embodiments, the TSO comprises a 3′ end region comprising rGrG+G, wherein rG denotes riboguanosine and +G denotes locked nucleic acid (LNA) guanosine. In some embodiments, the TSO comprises a 3′ end region comprising at least one isoguanine (iG) or isocytosine (iC) residue. In some embodiments, the 5′ end region comprising at least one isoguanine or isocytosine residue comprises iGiC.

In some aspects, the present disclosure provides for a kit for detecting an RNA molecule, comprising: (a) a stem-loop primer that comprises: a 5′ hemiprobe segment configured to hybridize to a first end region of the RNA molecule; a stem-loop segment; and a 3′ hemiprobe segment configured to hybridize to a second end region of the RNA molecule; and (b) a template switch oligonucleotide (TSO) providing an exogenous template, wherein the TSO is configured for elongation of the RNA molecule when the RNA molecule is reverse-transcribed. In some embodiments, the kit further comprises instructions for detecting the RNA molecule using the stem-loop primer and the TSO. In some embodiments, the kit further comprises a ribonuclease (RNAse) inhibitor solution. In some embodiments, the kit further comprises a reverse transcriptase. In some embodiments, the kit further comprises a fluorophore-linked oligonucleotide. In some embodiments, the TSO comprises a 3′ end region comprising rGrG+G, wherein rG denotes riboguanosine and +G denotes locked nucleic acid (LNA) guanosine. In some embodiments, the TSO comprises a 3′ end region comprising at least one isoguanine (iG) or isocytosine (iC) residue. In some embodiments, the 3′ end region comprising at least one isoguanine or isocytosine residue comprises iGiC.

In some aspects, the present disclosure provides for a composition, comprising: (a) an RNA molecule; (b) a stem-loop primer that comprises: a 5′ hemiprobe segment configured to hybridize to a first end region of the RNA molecule; a stem-loop segment; and a 3′ hemiprobe segment configured to hybridize to a second end region of the RNA molecule, and (c) a template switch oligonucleotide (TSO) that provides an exogenous template, wherein the TSO is configured for elongation of the RNA molecule when the RNA molecule is reverse-transcribed. In some embodiments, the composition further comprises an RNAse inhibitor. In some embodiments, the composition further comprises a reverse transcriptase. In some embodiments, the composition further comprises a fluorophore-linked oligonucleotide. In some embodiments, the RNA molecule is fewer than 30 nucleotides in length. In some embodiments, the RNA molecule is an siRNA or miRNA. In some embodiments, the composition further comprises a cell or cellular RNA. In some embodiments, the cell or cellular RNA comprises RNA molecules at least partially complementary to the RNA molecule. In some embodiments, the TSO comprises a 3′ end region comprising rGrG+G, wherein rG denotes riboguanosine and +G denotes locked nucleic acid (LNA) guanosine. In some embodiments, the TSO comprises a 3′ end region comprising at least one isoguanine (iG) or isocytosine (iC) residue. In some embodiments, the 3′ end region comprising at least one isoguanine or isocytosine residue comprises iGiC.

In some aspects, the present disclosure provides for a method for processing a ribonucleic acid (RNA) molecule, comprising: (a) providing a reaction mixture configured to produce a complementary DNA (cDNA) molecule from said RNA molecule, wherein said reaction mixture comprises: (i) said RNA molecule; and (ii) a stem-loop primer that comprises: a 5′ hemiprobe segment configured to hybridize to a complementary first terminus of said RNA molecule; a stem-loop segment; and a 3′ hemiprobe segment configured to hybridize to a complementary second terminus of said RNA molecule, and (iii) a template switch oligonucleotide (TSO) providing an exogenous template configured for 5′ end elongation of said RNA molecule when said RNA molecule is reverse-transcribed. In some embodiments, said exogenous template comprises a sequence exogenous to said RNA molecule. In some embodiments, a complement of said exogenous template is not configured to bind to said RNA molecule. In some embodiments, said RNA molecule is a naturally-occurring RNA molecule. In some embodiments, said RNA molecule is obtained from a sample from a cell, a tissue, or an organism. In some embodiments, said exogenous template is exogenous to said cell, said tissue, or said organism. In some embodiments, said exogenous template is not configured to hybridize to a cellular RNA from said cell, said tissue, or said organism. In some embodiments, said reaction mixture further comprises a cell or cellular RNA. In some embodiments, said cell or cellular RNA comprises targeted RNA molecules complementary to said RNA molecule. In some embodiments, the method further comprises producing said cDNA molecule from said RNA molecule. In some embodiments, said stem-loop segment comprises a first synthetic primer hybridization sequence. In some embodiments, said exogenous template of said TSO comprises a reverse complement of a second synthetic primer hybridization sequence. In some embodiments, the method further comprises detecting said cDNA molecule via said first synthetic primer hybridization sequence and said second synthetic primer hybridization sequence. In some embodiments, said detecting further comprises quantitative PCR (qPCR), digital PCR (dPCR), sequencing, or array hybridization. In some embodiments, said detecting further comprises contacting said cDNA molecule with a probe oligo configured to hybridize to an internal region of said cDNA molecule. In some embodiments, said probe oligo is a hybridization probe or a hydrolysis probe. In some embodiments, said probe oligo is configured to hybridize a region of a sequence complementary to said RNA molecule within said cDNA molecule. In some embodiments, said probe oligo is configured to hybridize to a region of said stem-loop segment within said cDNA molecule. In some embodiments, said detecting further comprises combining in an additional reaction mixture under conditions suitable to extend said cDNA molecule: (i) said cDNA molecule; (ii) a forward primer configured to hybridize to said first synthetic primer hybridization sequence; and (iii) a reverse primer. In some embodiments, said reverse primer is configured to hybridize to said second synthetic primer hybridization sequence. In some embodiments, said TSO comprises a 3′ end comprising rGrG. In some embodiments, said TSO comprises a 3′ terminal locked nucleic acid guanine. In some embodiments, said stem-loop segment is non-overlapping with said 5′ hemiprobe segment and said 3′ hemiprobe segment. In some embodiments, said RNA molecule comprises or consists of fewer than 40, 39, 38, 37, 36, 35, 34, 33, 32, 31, 30, 29, 28, 27, 26, 25, 24, 23, 22, 21, 20, 19, 18, 17, 16, 14, 13, 12, 11, or 10 nucleotides. In some embodiments, said RNA molecule is a short interfering RNA (siRNA) or a microRNA (miRNA). In some embodiments, said RNA molecule comprises a synthetic sugar-modified oligonucleotide or a synthetic linkage between two nucleotides of said RNA molecule. In some embodiments, said RNA molecule comprises a phosphorothioate linkage, a 2′-fluororibonucleotide, a 2′-O-methylribonucleotide, or a 2′-O-methoxyethylribonucleotide. In some embodiments, said reaction mixture comprises a reverse transcriptase. In some embodiments, said reverse transcriptase comprises Maxima™ H Minus reverse transcriptase, SuperScript™ II reverse transcriptase, Superscript™ III reverse transcriptase, SuperScript™ IV reverse transcriptase, Template Switching RT enzyme mix, SMARTscribe reverse transcriptase, PrimeScript™ RT, or any combination thereof. In some embodiments, said 5′ hemiprobe comprises about 5 to about 20 nucleotides. In some embodiments, said 3′ hemiprobe comprises about 3 to about 10 nucleotides. In some embodiments, said stem-loop segment comprises about 20 to about 80 nucleotides. In some embodiments, said first synthetic primer hybridization sequence comprises a sequence according to 5′-GGACAGAAACATAACATCAGAGT-3′ (SEQ ID NO: 5) or a sequence having at least 90%, 91%, 92%, 93%, 94%, 95%, 96%, 97%, 98%, 99%, or 100% sequence identity thereto. In some embodiments, said second synthetic primer hybridization sequence comprises a sequence according to 5′-CAGTGGTATCAACGCAG-3′ (SEQ ID NO: 6) or a sequence having at least 90%, 91%, 92%, 93%, 94%, 95%, 96%, 97%, 98%, 99%, or 100% sequence identity thereto.

In some aspects, the present disclosure provides for a method for processing an RNA molecule, comprising: (a) providing in a reaction mixture under conditions suitable for extending said RNA molecule: (i) said RNA molecule, which comprises a synthetic oligonucleotide or a synthetic linkage between two nucleotides of said RNA molecule; and (ii) a stem-loop primer that comprises: a 5′ hemiprobe segment configured to hybridize to a complementary first terminus of said RNA molecule; a stem-loop segment comprising a first synthetic primer hybridization sequence; and a 3′ hemiprobe segment configured to hybridize to a complementary second terminus of said RNA molecule; and (b) extending said stem-loop primer according to said RNA molecule under conditions suitable to incorporate a 3′ region comprising a second synthetic primer hybridization. sequence, thereby generating a cDNA molecule from said RNA molecule and said stem-loop primer. In some embodiments, the method further comprises detecting said cDNA molecule via said first synthetic primer hybridization sequence and said second synthetic primer hybridization sequence. In some embodiments, a complement of said second synthetic primer hybridization sequence is not configured to bind to said RNA molecule. In some embodiments, said RNA molecule is obtained from a cell, a tissue, or an organism. In some embodiments, said second synthetic primer hybridization sequence is exogenous to said cell, said tissue, or said organism. In some embodiments, a complement of said second synthetic primer hybridization sequence is not configured to hybridize to a cellular RNA from said cell, said tissue, or said organism. In some embodiments, said reaction mixture further comprises a cell or cellular RNA. In some embodiments, said cell or cellular RNA comprises targeted RNA molecules complementary to said RNA molecule. In some embodiments, the method further comprises detecting said cDNA molecule via said first synthetic primer hybridization sequence and said second synthetic primer hybridization sequence. In some embodiments, said detecting further comprises quantitative PCR (qPCR), digital PCR (dPCR), sequencing, or array hybridization. In some embodiments, said detecting further comprises contacting said cDNA molecule with a probe oligo configured to hybridize to an internal region of said cDNA molecule. In some embodiments, said probe oligo is a hybridization probe or a hydrolysis probe. In some embodiments, said probe oligo is configured to hybridize a region of a sequence complementary to said RNA molecule within said cDNA molecule. In some embodiments, said probe oligo is configured to hybridize to a region of said stem-loop segment within said cDNA molecule. In some embodiments, said detecting further comprises combining in an additional reaction mixture under conditions suitable to extend said cDNA molecule: (i) said cDNA molecule; (ii) a forward primer configured to hybridize to said first synthetic primer hybridization sequence; and (iii) a reverse primer. In some embodiments, said reverse primer is configured to hybridize to said second synthetic primer hybridization sequence. In some embodiments, said TSO comprises a 3′ end comprising rGrG. In some embodiments, said TSO comprises a 3′ terminal locked nucleic acid guanine. In some embodiments, said stem-loop segment is non-overlapping with said 5′ hemiprobe segment and said 3′ hemiprobe segment. In some embodiments, said RNA molecule comprises or consists of fewer than 40, 39, 38, 37, 36, 35, 34, 33, 32, 31, 30, 29, 28, 27, 26, 25, 24, 23, 22, 21, 20, 19, 18, 17, 16, 14, 13, 12, 11, or 10 nucleotides. In some embodiments, said RNA molecule is a short interfering RNA (siRNA) or a microRNA (miRNA). In some embodiments, said RNA molecule comprises a synthetic sugar-modified oligonucleotide or a synthetic linkage between two nucleotides of said RNA molecule. In some embodiments, said RNA molecule comprises a phosphorothioate linkage, a 2′-fluororibonucleotide, a 2′-O-methylribonucleotide, or a 2′-O-methoxyethylribonucleotide. In some embodiments, said reaction mixture comprises a reverse transcriptase. In some embodiments, said reverse transcriptase comprises Maxima™ H Minus reverse transcriptase, SuperScript™ II reverse transcriptase, Superscript™ III reverse transcriptase, SuperScript™ IV reverse transcriptase, Template Switching RT enzyme mix, SMARTscribe reverse transcriptase, PrimeScript™ RT, or any combination thereof. In some embodiments, said 5′ hemiprobe comprises about 5 to about 20 nucleotides. In some embodiments, said 3′ hemiprobe comprises about 3 to about 10 nucleotides. In some embodiments, said stem-loop segment comprises about 20 to about 80 nucleotides. In some embodiments, said first synthetic primer hybridization sequence comprises a sequence according to 5′-GGACAGAAACATAACATCAGAGT-3′ (SEQ ID NO: 5) or a sequence having at least 90%, 91%, 92%, 93%, 94%, 95%, 96%, 97%, 98%, 99%, or 100% sequence identity thereto. In some embodiments, said second synthetic primer hybridization sequence comprises a sequence according to 5′-CAGTGGTATCAACGCAG-3′ (SEQ ID NO: 6) or a sequence having at least 90%, 91%, 92%, 93%, 94%, 95%, 96%, 97%, 98%, 99%, or 100% sequence identity thereto.

In some aspects, the present disclosure provides for a kit for detecting an RNA molecule, comprising: (a) a stem-loop primer that comprises: a 5′ hemiprobe segment configured to hybridize to a complementary first terminus of said RNA molecule; a stem-loop segment; and a 3′ hemiprobe segment configured to hybridize to a complementary second terminus of said RNA molecule; (b) a template switch oligonucleotide (TSO) providing an exogenous template configured for 5′ end elongation of said RNA molecule; and (c) instructions for detecting said RNA molecule using said stem-loop primer and said template switch oligonucleotide. In some embodiments, the kit further comprises an RNAse inhibitor solution. In some embodiments, the kit further comprises a reverse transcriptase. In some embodiments, the kit further comprises a fluorophore-linked oligonucleotide. In some embodiments, said exogenous template comprises a sequence exogenous to said RNA molecule. In some embodiments, a complement of said exogenous template is not configured to bind to said RNA molecule. In some embodiments, said RNA molecule is a naturally-occurring RNA molecule. In some embodiments, said RNA molecule is obtained from a sample from a cell, a tissue, or an organism. In some embodiments, said exogenous template is exogenous to said cell, said tissue, or said organism. In some embodiments, said exogenous template is not configured to hybridize to a cellular RNA from said cell, said tissue, or said organism. In some embodiments, said reaction mixture further comprises a cell or cellular RNA. In some embodiments, said cell or cellular RNA comprises targeted RNA molecules complementary to said RNA molecule. In some embodiments, the method further comprises producing said cDNA molecule from said RNA molecule. In some embodiments, said stem-loop segment comprises a first synthetic primer hybridization sequence. In some embodiments, said exogenous template of said TSO comprises a reverse complement of a second synthetic primer hybridization sequence. In some embodiments, the method further comprises detecting said cDNA molecule via said first synthetic primer hybridization sequence and said second synthetic primer hybridization sequence. In some embodiments, said detecting further comprises quantitative PCR (qPCR), digital PCR (dPCR), sequencing, or array hybridization. In some embodiments, said detecting further comprises contacting said cDNA molecule with a probe oligo configured to hybridize to an internal region of said cDNA molecule. In some embodiments, said probe oligo is a hybridization probe or a hydrolysis probe. In some embodiments, said probe oligo is configured to hybridize a region of a sequence complementary to said RNA molecule within said cDNA molecule. In some embodiments, said probe oligo is configured to hybridize to a region of said stem-loop segment within said cDNA molecule. In some embodiments, said detecting further comprises combining in an additional reaction mixture under conditions suitable to extend said cDNA molecule: (i) said cDNA molecule; (ii) a forward primer configured to hybridize to said first synthetic primer hybridization sequence; and (iii) a reverse primer. In some embodiments, said reverse primer is configured to hybridize to said second synthetic primer hybridization sequence. In some embodiments, said TSO comprises a 3′ end comprising rGrG. In some embodiments, said TSO comprises a 3′ terminal locked nucleic acid guanine. In some embodiments, said stem-loop segment is non-overlapping with said 5′ hemiprobe segment and said 3′ hemiprobe segment. In some embodiments, said RNA molecule comprises or consists of fewer than 40, 39, 38, 37, 36, 35, 34, 33, 32, 31, 30, 29, 28, 27, 26, 25, 24, 23, 22, 21, 20, 19, 18, 17, 16, 14, 13, 12, 11, or 10 nucleotides. In some embodiments, said RNA molecule is a short interfering RNA (siRNA) or a microRNA (miRNA). In some embodiments, said RNA molecule comprises a synthetic sugar-modified oligonucleotide or a synthetic linkage between two nucleotides of said RNA molecule. In some embodiments, said RNA molecule comprises a phosphorothioate linkage, a 2′-fluororibonucleotide, a 2′-O-methylribonucleotide, or a 2′-O-methoxyethylribonucleotide. In some embodiments, said reaction mixture comprises a reverse transcriptase. In some embodiments, said reverse transcriptase comprises Maxima™ H Minus reverse transcriptase, SuperScript™ II reverse transcriptase, Superscript™ III reverse transcriptase, SuperScript™ IV reverse transcriptase, Template Switching RT enzyme mix, SMARTscribe reverse transcriptase, PrimeScript™ RT, or any combination thereof. In some embodiments, said 5′ hemiprobe comprises about 5 to about 20 nucleotides. In some embodiments, said 3′ hemiprobe comprises about 3 to about 10 nucleotides. In some embodiments, said stem-loop segment comprises about 20 to about 80 nucleotides. In some embodiments, said first synthetic primer hybridization sequence comprises a sequence according to 5′-GGACAGAAACATAACATCAGAGT-3′ (SEQ ID NO: 5) or a sequence having at least 90%, 91%, 92%, 93%, 94%, 95%, 96%, 97%, 98%, 99%, or 100% sequence identity thereto. In some embodiments, said second synthetic primer hybridization sequence comprises a sequence according to 5′-CAGTGGTATCAACGCAG-3′ (SEQ ID NO: 6) or a sequence having at least 90%, 91%, 92%, 93%, 94%, 95%, 96%, 97%, 98%, 99%, or 100% sequence identity thereto.

In some aspects, the present disclosure provides for a mixture, comprising: (a) an RNA molecule; and (b) a stem-loop primer that comprises: a 5′ hemiprobe segment configured to hybridize to a complementary first terminus of said RNA molecule; a stem-loop segment; and a 3′ hemiprobe segment configured to hybridize to a complementary second terminus of said RNA molecule, and (c) a template switch oligonucleotide (TSO) providing an exogenous template configured for 5′ end elongation of said RNA molecule when said RNA molecule is reverse-transcribed. In some embodiments, the mixture further comprises an RNAse inhibitor. In some embodiments, the mixture further comprises a reverse transcriptase. In some embodiments, the mixture further comprises a fluorophore-linked oligonucleotide. In some embodiments, said RNA molecule consists of fewer than 30 nucleotides. In some embodiments, said RNA molecule is an siRNA or miRNA. In some embodiments, the mixture further comprises a cell or cellular RNA. In some embodiments, said cell or cellular RNA comprises targeted RNA molecules complementary to said RNA molecule. In some embodiments, said TSO comprises a 3′ end comprising rGrG+G, wherein rG denotes riboguanosine and +G denotes locked nucleic acid (LNA) guanosine. In some embodiments, said TSO comprises a 5′ end comprising at least one isoguanine (iG) or isocytosine (iC) residue. In some embodiments, said 5′ end comprising at least one isoguanine or isocytosine residue comprises iGiC. In some embodiments, said exogenous template comprises a sequence exogenous to said RNA molecule. In some embodiments, a complement of said exogenous template is not configured to bind to said RNA molecule. In some embodiments, said RNA molecule is a naturally-occurring RNA molecule. In some embodiments, said RNA molecule is obtained from a sample from a cell, a tissue, or an organism. In some embodiments, said exogenous template is exogenous to said cell, said tissue, or said organism. In some embodiments, said exogenous template is not configured to hybridize to a cellular RNA from said cell, said tissue, or said organism. In some embodiments, said reaction mixture further comprises a cell or cellular RNA. In some embodiments, said cell or cellular RNA comprises targeted RNA molecules complementary to said RNA molecule. In some embodiments, the method further comprises producing said cDNA molecule from said RNA molecule. In some embodiments, said stem-loop segment comprises a first synthetic primer hybridization sequence. In some embodiments, said exogenous template of said TSO comprises a reverse complement of a second synthetic primer hybridization sequence. In some embodiments, the method further comprises detecting said cDNA molecule via said first synthetic primer hybridization sequence and said second synthetic primer hybridization sequence. In some embodiments, said detecting further comprises quantitative PCR (qPCR), digital PCR (dPCR), sequencing, or array hybridization. In some embodiments, said detecting further comprises contacting said cDNA molecule with a probe oligo configured to hybridize to an internal region of said cDNA molecule. In some embodiments, said probe oligo is a hybridization probe or a hydrolysis probe. In some embodiments, said probe oligo is configured to hybridize a region of a sequence complementary to said RNA molecule within said cDNA molecule. In some embodiments, said probe oligo is configured to hybridize to a region of said stem-loop segment within said cDNA molecule. In some embodiments, said detecting further comprises combining in an additional reaction mixture under conditions suitable to extend said cDNA molecule: (i) said cDNA molecule; (ii) a forward primer configured to hybridize to said first synthetic primer hybridization sequence; and (iii) a reverse primer. In some embodiments, said reverse primer is configured to hybridize to said second synthetic primer hybridization sequence. In some embodiments, said TSO comprises a 3′ end comprising rGrG. In some embodiments, said TSO comprises a 3′ terminal locked nucleic acid guanine. In some embodiments, said stem-loop segment is non-overlapping with said 5′ hemiprobe segment and said 3′ hemiprobe segment. In some embodiments, said RNA molecule comprises or consists of fewer than 40, 39, 38, 37, 36, 35, 34, 33, 32, 31, 30, 29, 28, 27, 26, 25, 24, 23, 22, 21, 20, 19, 18, 17, 16, 14, 13, 12, 11, or 10 nucleotides. In some embodiments, said RNA molecule is a short interfering RNA (siRNA) or a microRNA (miRNA). In some embodiments, said RNA molecule comprises a synthetic sugar-modified oligonucleotide or a synthetic linkage between two nucleotides of said RNA molecule. In some embodiments, said RNA molecule comprises a phosphorothioate linkage, a 2′-fluororibonucleotide, a 2′-O-methylribonucleotide, or a 2′-O-methoxyethylribonucleotide. In some embodiments, said reaction mixture comprises a reverse transcriptase. In some embodiments, said reverse transcriptase comprises Maxima™ H Minus reverse transcriptase, SuperScript™ II reverse transcriptase, Superscript™ III reverse transcriptase, SuperScript™ IV reverse transcriptase, Template Switching RT enzyme mix, SMARTscribe reverse transcriptase, PrimeScript™ RT, or any combination thereof. In some embodiments, said 5′ hemiprobe comprises about 5 to about 20 nucleotides. In some embodiments, said 3′ hemiprobe comprises about 3 to about 10 nucleotides. In some embodiments, said stem-loop segment comprises about 20 to about 80 nucleotides. In some embodiments, said first synthetic primer hybridization sequence comprises a sequence according to 5′-GGACAGAAACATAACATCAGAGT-3′ (SEQ ID NO: 5) or a sequence having at least 90%, 91%, 92%, 93%, 94%, 95%, 96%, 97%, 98%, 99%, or 100% sequence identity thereto. In some embodiments, said second synthetic primer hybridization sequence comprises a sequence according to 5′-CAGTGGTATCAACGCAG-3′ (SEQ ID NO: 6) or a sequence having at least 90%, 91%, 92%, 93%, 94%, 95%, 96%, 97%, 98%, 99%, or 100% sequence identity thereto.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### Color Drawings

The patent or application file contains at least one drawing executed in color. Copies of this patent or patent application publication with color drawing(s) will be provided by the office upon request and payment of the necessary fee.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

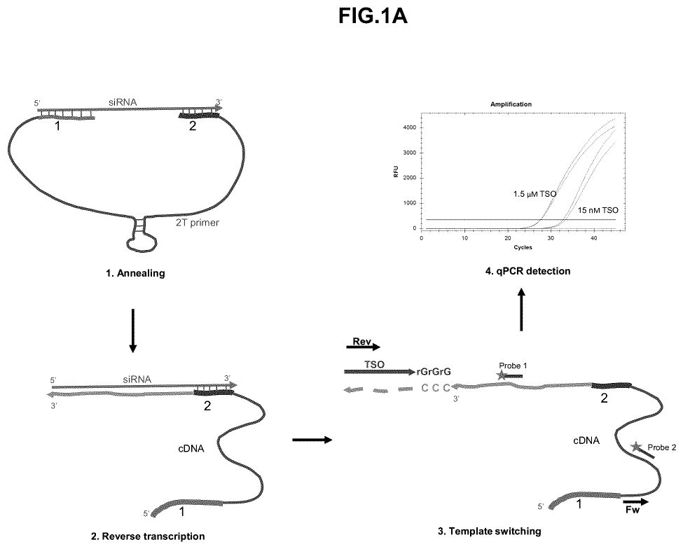

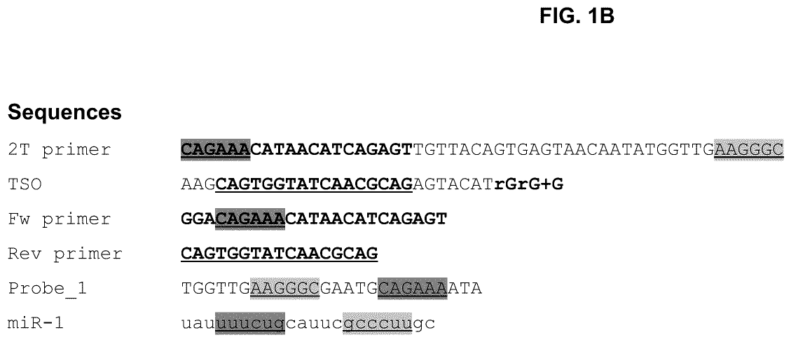

FIG. 1A depicts a schematic illustration of the two-tailed processing method according to the current disclosure. In the method, a two-tailed (2T) primer first binds to the RNA (e.g. siRNA) and primes cDNA synthesis. Upon reaching the end of the RNA, the reverse transcriptase switches template to the template switching oligonucleotide (TSO), thereby adding a priming site for a reverse qPCR primer (“Rev”). The result is read out by qPCR, using the Rev primer together with a forward primer (Fw) that anneals in the 2-tailed (2T) sequence and an RNA-specific probe (Probe 1) (e.g. an siRNA-specific probe). Alternatively, a pan-RNA (e.g. pan-siRNA) probe that binds in the universal 2T sequence can be used (Probe 2).

FIG. 1B depicts sequences of oligonucleotides used in methods according to the current disclosure. Grey highlight: 5′ and 3′ hemiprobe segments. Bold underlined: Rev primer sequence. rN: ribonucleotide. +N: locked nucleic acid.: nucleotide A, U, C or G. Figure discloses SEQ ID NOS 3-7 and 1, respectively, in order of appearance.

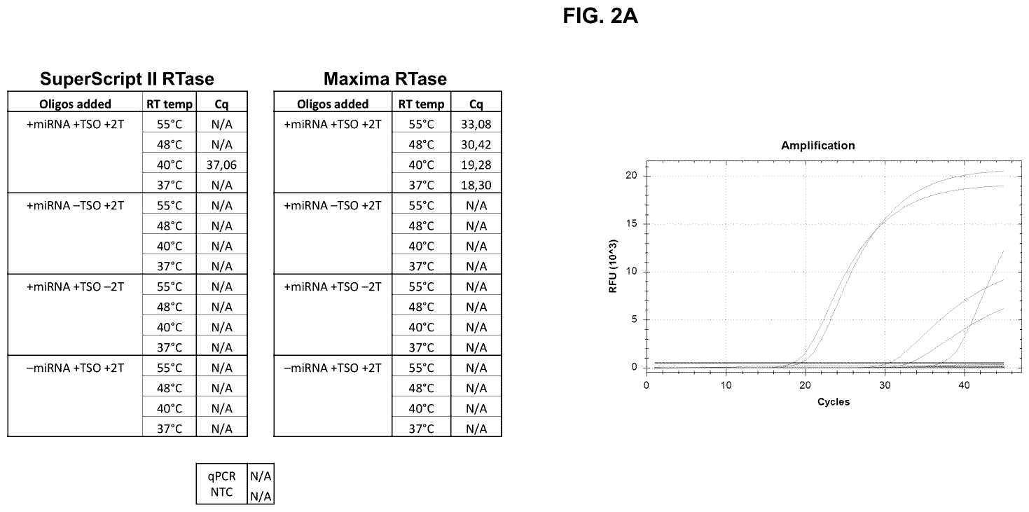

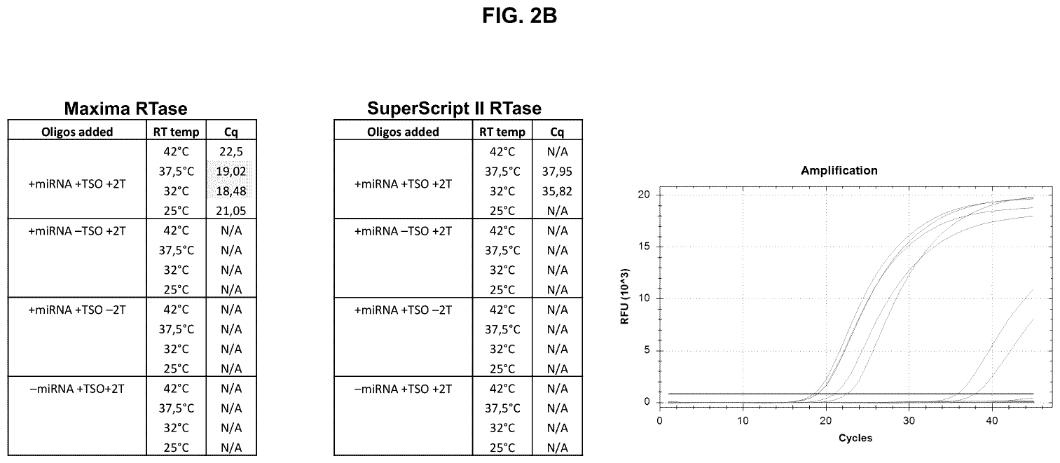

FIGS. 2A and 2B depict quantitation of small RNAs (sRNAs, e.g. siRNAs, miRNAs) using different reverse transcriptases and different reverse transcriptase reaction temperatures according to methods of the disclosure. Shown are charts of reverse transcriptase step temp (“RT temp”) and quantification cycle (Cq) alongside a qPCR trace of RFU versus cycle. RT reactions were run at 37° C., 40° C., 48° C. and 55° C. for FIG. 2A, or 25° C., 32° C., 37.5° C. and 42° C. for FIG. 2B. Two RTases were tested: SuperScript II and Maxima H Minus. RT negative controls contained either no TSO (“-TSO”), no 2T primer (“-2T”) or no sRNA (“-miRNA”). qPCR NTC: water was added instead of RT product to the qPCR reaction.

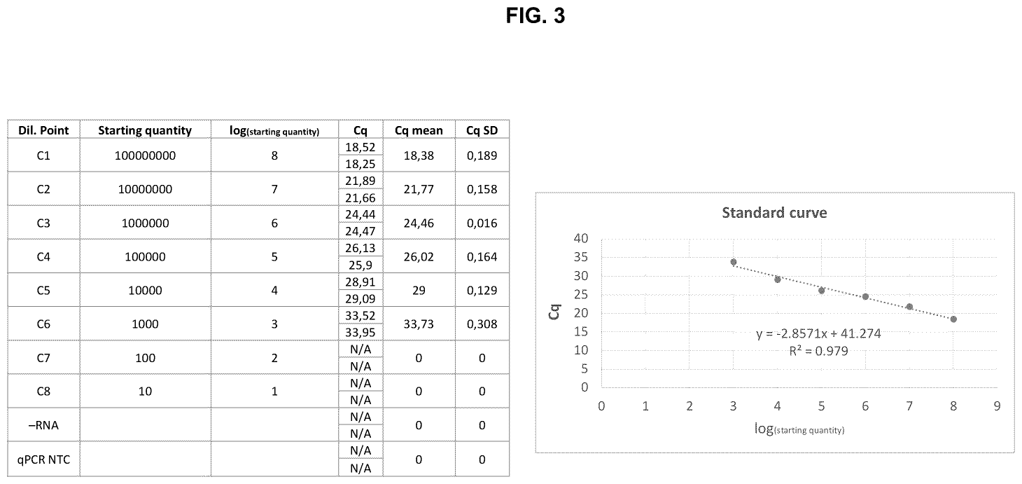

FIG. 3 depicts results of experiments demonstrating sensitivity for quantitation of RNAs (e.g. sRNA, siRNA, miRNA) using methods according to the disclosure. Shown is a chart of the relationship between quantity of RNA (e.g. sRNA, siRNA, miRNA) and quantitation cycle (Cq) and a graph showing the derived standard curve and associated regression equation. The sRNA was diluted ten-fold in TE-LPA buffer to generate an eight-point standard curve. Duplicate samples were set up at the RT step. The sRNA was omitted in the negative RT control (“-RNA”) whereas the cDNA was omitted in the qPCR control (“qPCR NTC”). The assay detected down to 1000 molecules.

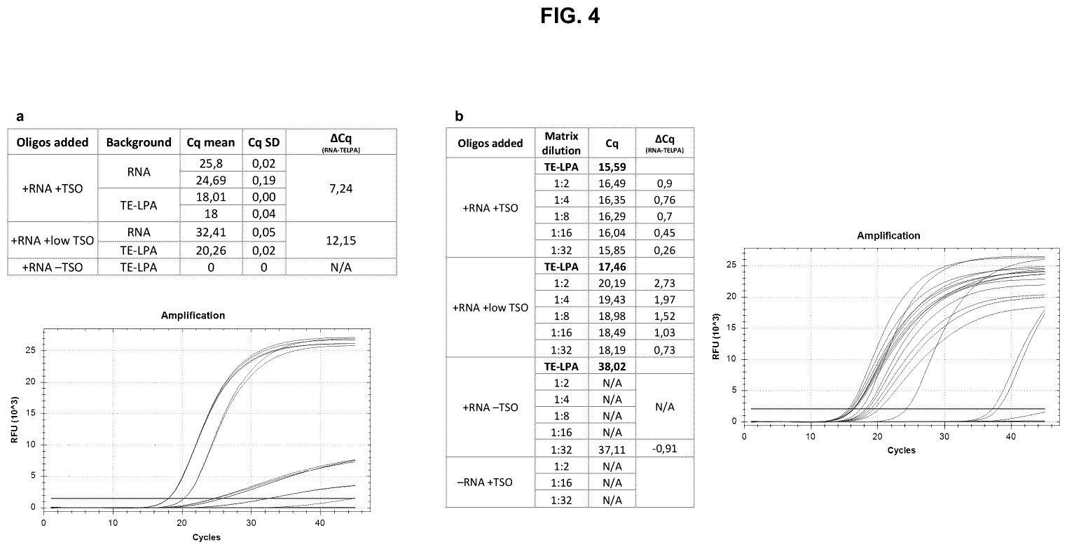

FIG. 4 demonstrates resistance of methods according to the disclosure to interference/inhibition by bulk cellular RNAs. Shown is a chart showing Cq under different conditions alongside qPCR graphs of the relevant experiments. Robustness toward inhibition by cellular RNAs was performed by spiking siRNA_1 into different concentrations of mouse liver RNA. Panel a: 2T-TS in undiluted mouse RNA matrix. Values are averages of duplicates set up at the RT step. The Cq delay and the flat amplification curves clearly indicate inhibition in undiluted matrix. Panel b: the inhibition effect is overcome by diluting the matrix RNA (smallest ΔCq at 1:32 dilution). TE-LPA: buffer used for matrix dilution.

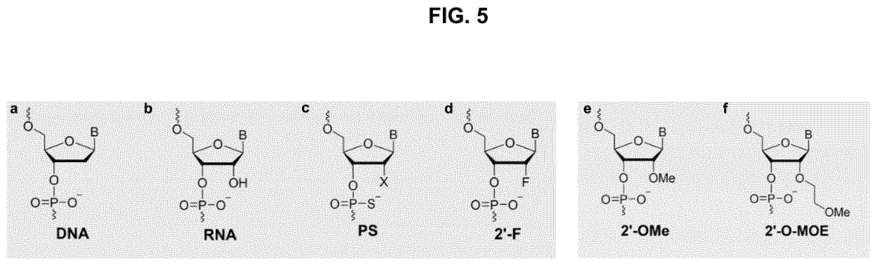

FIG. 5 depicts tested sRNA nucleotide modifications compatible with methods according to the disclosure. Natural DNA (a) and RNA (b) are displayed for reference. Modifications tested here were (c) PS (phosphorothioate), (d) 2′-F (2′-fluoro), (e) 2′-O-Me (2′-O-methyl) and (f) 2′-O-MOE (2′-O-Methoxyethyl). PS is a phosphonate group modification, whereas 2′-F, 2′-O-Me, and 2′-O-MOE are modifications of the ribose moiety

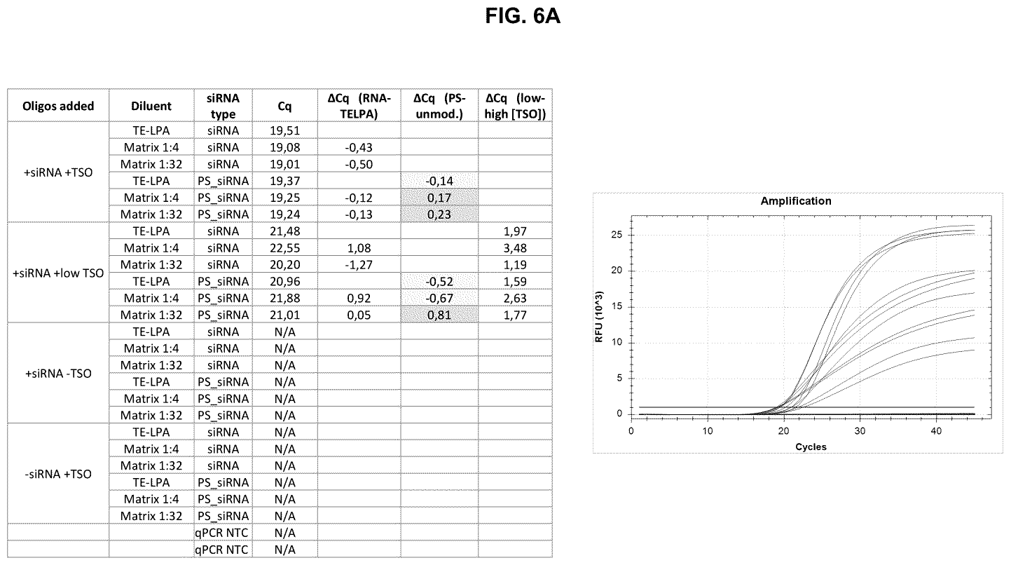

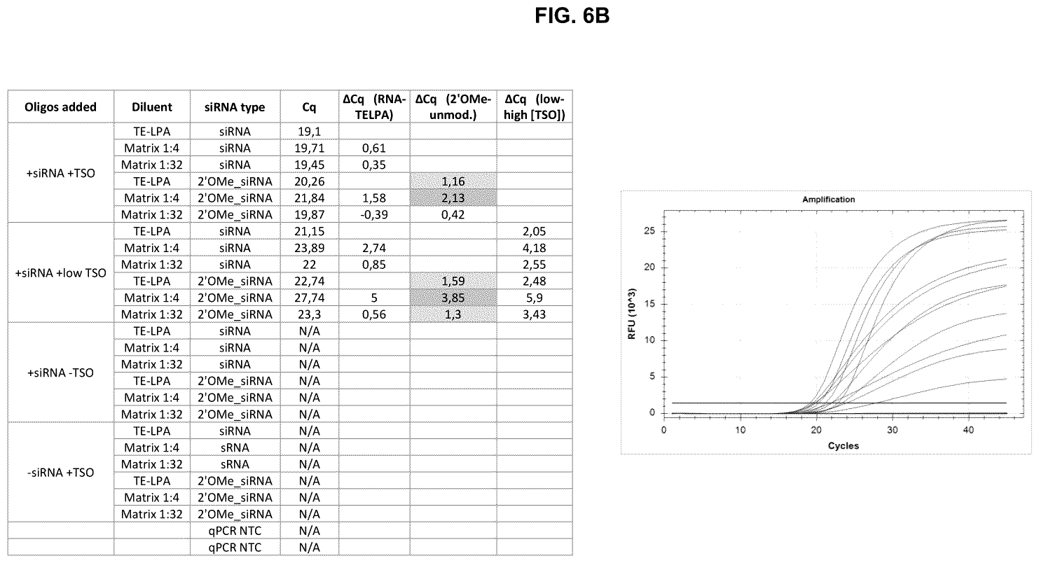

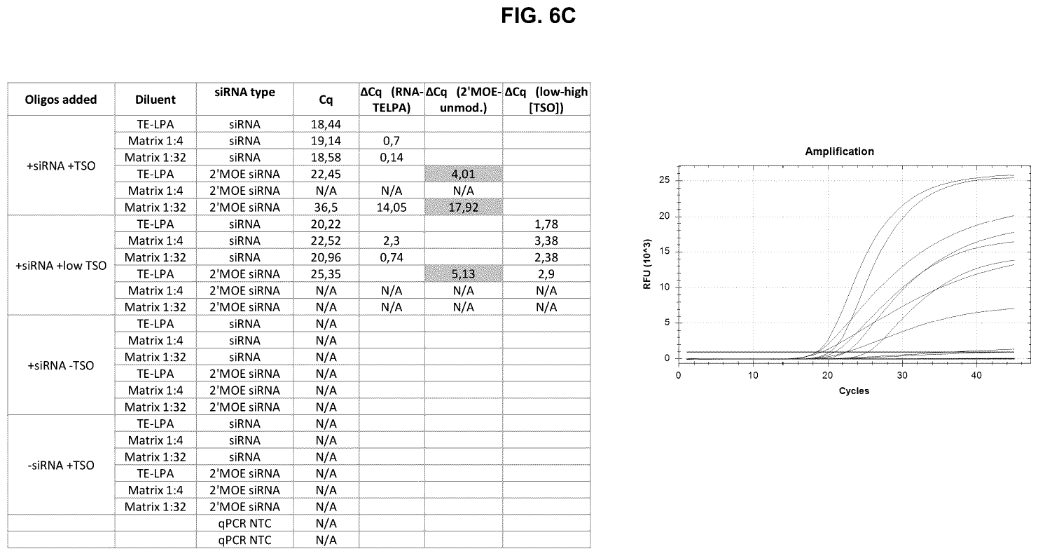

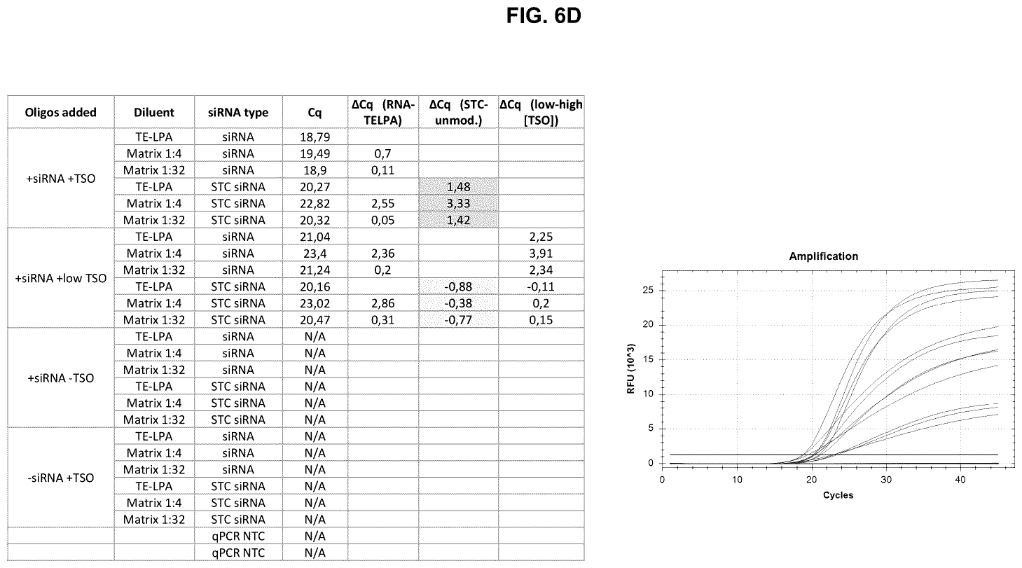

FIGS. 6A, 6B, 6C, and 6D depict performance of methods according to the disclosure on chemically-modified RNAs (e.g. chemically-modified siRNAs). Displayed are Cq data and amplification plots from qPCR readout of the 2T-TS assay targeting modified siRNAs. FIG. 6A shows results for siRNA with PS (phosphorothioate) modification at positions 19 and 20. FIG. 6B shows results siRNA with 2′-O-me modification at residues 7 and 17. FIG. 6C shows results for siRNA with 2′-O-MOE modification at positions 7 and 18. FIG. 6D shows results for fully modified siRNA (alternating 2′-F and 2′-O-me nucleotides). The data indicate the method of FIG. 1 can detect PS-carrying siRNA without effects on assay sensitivity (a). There is a minor decrease in sensitivity on 2′-O-me siRNA (FIGS. 6B and 6D). As documented previously, RT was less efficient over 2′-O-MOE residues (FIG. 6C).

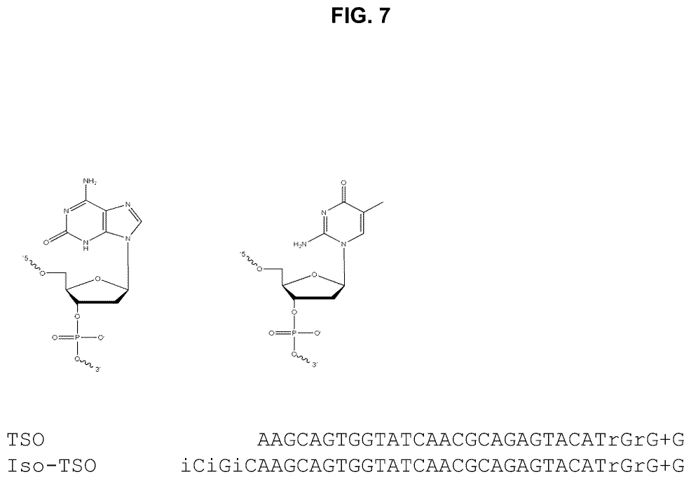

FIG. 7 depicts structures of iso-nucleotides (e.g., for addition to a TSO 5′ end) useful with methods of the disclosure to prevent artifact formation. Figure discloses SEQ ID NOS 4 and 8, respectively, in order of appearance.

### DETAILED DESCRIPTION

### Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” generally refers to an amount that is near the stated amount by about 10%, 5%, or 1%, including increments therein. For example, “about” or “approximately” can mean a range including the particular value and ranging from 10% below that particular value and spanning to 10% above that particular value.

The practice of some methods disclosed herein employ, unless otherwise indicated, techniques of immunology, biochemistry, chemistry, molecular biology, microbiology, cell biology, genomics and recombinant DNA. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)) (which is entirely incorporated by reference herein).

The terms “polynucleotide,” “nucleic acid,” and “oligonucleotide,” as used herein, generally refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, intergenic DNA, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), small nucleolar RNA, ribozymes, cDNA, recombinant polynucleotides, branched polynucleotides, plasmids, vectors, isolated DNA of any sequence, isolated RNA of any sequence, nucleic acid probes, adapters, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component, tag, reactive moiety, or binding partner. Polynucleotide sequences, when provided, are listed in the 5′ to 3′ direction, unless stated otherwise. A polynucleotide can comprise a non-natural nucleotide or a non-natural linkage. A polynucleotide can comprise a synthetic sugar-modified oligonucleotide (e.g. a 2′-fluororibonucleotide, a 2′-O-methylribonucleotide, a 2′-O-methoxyethylribonucleotide, a locked nucleic acid (LNA), an unlocked nucleic acid (UNA), a bridged nucleic acid (BNA) or a glycol nucleic acid (GNA), or a synthetic linkage between two nucleotides (e.g. a phosphorothioate linkage, a methylphosphonate linkage, a methoxypropyl-phosphonate linkage, or a peptide linkage.

As used herein, the term “short-interfering RNA” or “siRNA” generally refers to a double stranded nucleic acid in which each strand comprises RNA, RNA analog(s) or RNA and DNA. In some embodiments, an siRNA comprises between 19 and 23 nucleotides or comprises 21 nucleotides. In some cases, the siRNA comprises 2 bp overhangs on the 3′ ends of each strand such that the duplex region in the siRNA comprises 17-21 nucleotides, or 19 nucleotides. In some embodiments, the antisense strand of the siRNA is sufficiently complementary with a sequence of an mRNA targeted for disruption.

“Hybridizes”, as used herein, generally refers to a reaction in which one or more polynucleotides interact to form a complex that is stabilized via hydrogen bonding between the bases of the nucleotide residues. The hydrogen bonding may occur by Watson Crick base pairing, Hoogstein binding, or in any other sequence sensitive or specific manner. The complex may comprise two strands forming a duplex structure, three or more strands forming a multi stranded complex, a single self-hybridizing strand, or any combination of these. A hybridization reaction may constitute a step in a more extensive process, such as the initiation of a PCR, or the enzymatic cleavage of a polynucleotide by a ribozyme. A first sequence that can be stabilized via hydrogen bonding with the bases of the nucleotide residues of a second sequence can generally be said to be “hybridizable” to the second sequence. In such a case, the second sequence can also be said to be hybridizable to the first sequence.

“Complement,” “complements,” “complementary,” and “complementarity,”, as used herein, generally refer to a sequence that is fully complementary to and hybridizable to the given sequence. In some cases, a first sequence that is hybridizable to a second sequence or set of second sequences is specifically or selectively hybridizable to the second sequence or set of second sequences, such that hybridization to the second sequence or set of second sequences is used. Hybridizable sequences can share a degree of sequence complementarity over all or a portion of their respective lengths, such as between 25%-100% complementarity, including at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, and 100% sequence complementarity.

As used herein, the terms “amplify,” “amplifies,” “amplified,” “amplification,” and “amplicon” generally refer to any method for replicating a nucleic acid with the use of a primer-dependent polymerase and/or those processes' products. In some cases, the amplification is effected by PCR using a pair of primers, comprising a first and second primer as described above. Amplified products can be subjected to subsequence analyses, including but not limited to melting curve analysis, nucleotide sequencing, single-strand conformation polymorphism assay, allele-specific oligonucleotide hybridization, Southern blot analysis, and restriction endonuclease digestion.

Amplification products may be detected by the use of a probe. As used herein, the term “probe” generally refers to a polynucleotide that carries a detectable member and has complementarity to a target nucleic acid, thus being able to hybridize with said target and be detected by said detectable member. In certain embodiments, a probe may include Watson-Crick bases or modified bases. Modified bases include, but are not limited to, the AEGIS bases described, e.g., in U.S. Pat. Nos. 5,432,272; 5,965,364; and 6,001,983, each of which are entirely incorporated by reference herein. In certain aspects, bases are joined by a natural phosphodiester bond or a different chemical linkage. Different chemical linkages include, but are not limited to, a peptide bond, an LNA linkage, or a phosphorothioate linkage.

Amplification can be performed by any suitable method. The nucleic acids may be amplified by polymerase chain reaction (PCR), as described in, for example, U.S. Pat. Nos. 5,928,907 and 6,015,674, each of which are incorporated by reference herein for any purpose. Other methods of nucleic acid amplification may include, for example, ligase chain reaction, oligonucleotide ligations assay, and hybridization assay, as described in greater detail in U.S. Pat. Nos. 5,928,907 and 6,015,674, each of which are incorporated by reference herein in their entirety. Methods can involve real-time optical detection systems described in greater detail in, for example, U.S. Pat. Nos. 5,928,907 and 6,015,674, each which are incorporated by reference herein. Other amplification methods that can be used according to some methods of the disclosure include those described in U.S. Pat. Nos. 5,242,794; 5,494,810; 4,988,617; and 6,582,938, each of which are incorporated herein in their entirety.

As used herein, the term “stem-loop” generally refers to a nucleic acid structure comprising a double-stranded stem and an intervening loop stabilized by intramolecular base-pairing through hydrogen bonding of a plurality of nucleotides single stranded nucleic acid molecule (e.g. a “hairpin”). Such a stem loop structure can have an increased melting point (Tm) relative to a non-intramolecularly-paired nucleic acid.

The term “hydrolysis probe” as used herein generally refers to an oligonucleotide which can be enzymatically hydrolyzed. In some embodiments, a “hydrolysis probe” generally refers to an oligonucleotide which can be hydrolyzed by the 5′- to 3′ exonuclease activity of a DNA polymerase (e.g. Taq DNA polymerase) during a PCR reaction. In some embodiments, a “hydrolysis probe” of the present invention comprises a TaqMan probe, e.g. a sequence-specific oligonucleotide carrying both a fluorophore and a quencher moiety, in which the fluorophore and the quencher moiety are attached to the oligonucleotide as such that the proximity of the fluorophore (e.g. the fluorescent reporter or the fluorescent label) to the quencher prevents the reporter from fluorescing. TaqMan probes can be designed as such that the fluorophore is attached at or near the 5′-end of the oligonucleotide probe, while the quencher is located at or near the 3′-end. During the combined annealing/extension phase of a polynucleotide chain reaction (PCR), the probe can be cleaved by the 5′- to 3′ exonuclease activity of a DNA polymerase, thereby separating the fluorophore and the quencher moieties, with the consequence that the fluorescence reporter can emit a fluorescence signal which can then be measured. The detectable fluorescence can be proportional to the amount of accumulated PCR product.

In some cases, a fluorophore (e.g. a fluorescent reporter or a florescent label) attached to a hydrolysis probe may be selected from, but is not limited to, the group of fluorescein dyes such as carboxyfluorescein (FAM), 6-carboxy-4′,5′-dichloro-2′7′-dimethoxyfluorescein (JOE), fluorescein isothiocyanate (FITC), tetrachlorofluorescein (TET), or 5′-Hexachloro-Fluorescein-CE Phosphoramidite (HEX); rhodamine dyes such as, e.g., carboxy-X-rhodamine (ROX), Texas Red and tetramethylrhodamine (TAMRA), cyanine dyes such as pyrylium cyanine dyes, DY548, Quasar 570, or dyes such as Cy3, Cy5, Alexa 568, or alike. The choice of the fluorescent label is typically determined by its spectral properties and by the availability of equipment for imaging. In some cases, any suitable fluorophore is used.

A quencher generally refers to a molecule which absorbs energy transferred from a donor molecule (e.g. a fluorescent reporter). In some cases, the donor molecule transfers energy to the quencher, and the donor returns to the ground state and generates the excited state of the quencher. Fluorescence quenching efficiency can depend on the distance between the donor and the acceptor molecule. In some cases, a quencher is one of a group of fluorescent dyes, for example, fluorescein as the reporter and rhodamine as the quencher (FAMJTAMRA probes). In some cases, the quencher is TAMRA (tetramethyl-rhodamine) which can be used to lower the emission of the reporter dye. Due to its properties TAMRA can be suitable as a quencher for FAM (carboxyfluorescein), HEX (hexachlorofluorescein), TET (tetrachloro-fluorescein), JOE (5′-Dichloro-dimethoxy-fluorescein) and Cy3-dyes (cyanine). Alternatively, a “quencher” can be a non-fluorescent (e.g. dark) quencher which can enable multiplexing. Dark quenchers which may be applicable in the context of the present invention include, but are not limited to, agents such as, e.g. DABCYL (4-[[4-(dimethylamino)-phenyl]-azo]-benzoic acid) which quenches dyes in a range of from 380 to 530 nm, the Eclipse Quencher (4-[[2-chloro-4-nitro-phenyl]-azo]-aniline; which has an absorption maximum at 530 nm and efficiently quenches over a spectrum from 520 to 670 nm, or Black Hole Quenchers, such as ([(4-(2-nitro-4-methyl-phenyl)-azo)-yl-((2-methoxy-5-methyl-phenyl)-azo)]-aniline) and BHQ-2 ([(4-(1-nitro-phenyl)-azo)-yl-((2,5-dimethoxy-phenyl)-azo)]-aniline) which are capable of quenching across the entire visible spectrum. These non-fluorescent acceptors can be applied as alternative to fluorescent acceptors in order to decrease background fluorescence and thus sensitivity.

As used herein, the term “hybridization probe” generally refers to a fragment of DNA or RNA of usually 15-25 nucleotide long which can be radioactively or fluorescently labeled. A hybridization probe can be used to detect the presence of nucleotide sequences in analyzed RNA or DNA that are complementary to the sequence in the probe.

### EXAMPLE EMBODIMENTS

In some aspects, the present disclosure provides for a method for processing a ribonucleic acid (RNA) molecule. In some cases, the RNA molecule is a short RNA molecule, a short interfering RNA (siRNA), a microRNA (miRNA), a transfer RNA, a ribosomal RNA, a short-hairpin RNA (shRNA), or a small nucleolar RNA. In some cases, said RNA molecule is a naturally-occurring RNA molecule. In some cases, said RNA molecule is obtained from a sample from cell, a tissue, or an organism. In some embodiments, said RNA molecule comprises a synthetic sugar-modified oligonucleotide or a synthetic linkage between two nucleotides of said RNA molecule. In some embodiments, said RNA molecule comprises a phosphorothioate linkage, a 2′-fluororibonucleotide, a 2′-O-methylribonucleotide, or a 2′-O-methoxyethylribonucleotide.

In some embodiments, the method for processing the ribonucleic acid (RNA) molecule comprises: providing a reaction mixture configured to produce a complementary DNA (cDNA) molecule from said RNA molecule, wherein said first reaction mixture comprises: (i) said RNA molecule; (ii) a stem-loop primer; and (iii) a template switch oligonucleotide (TSO). In some embodiments, the method further comprises (a) generating said cDNA molecule. In some embodiments, the method further comprises: (b) detecting said cDNA molecule. In some embodiments, said cDNA molecule is detected at least in part via a synthetic primer hybridization sequence contained within said TSO or said stem-loop primer. In some embodiments, said detecting further comprises quantitative PCR (qPCR), digital PCR (dPCR), sequencing, or array hybridization. In some embodiments, said detecting further comprises contacting said cDNA with a probe oligo configured to hybridize to an internal region of said cDNA. In some embodiments, said detecting further comprises contacting said cDNA with a probe oligo configured to hybridize to an internal region of said cDNA molecule. In some embodiments, said probe oligo is configured to hybridize a region of a sequence complementary to said RNA molecule within said cDNA molecule. In some embodiments, said probe oligo is a hybridization probe or hydrolysis probe. In some embodiments, said probe oligo is configured to hybridize to a region of said stem-loop within said cDNA molecule. In some embodiments, said detecting further comprises combining in said reaction mixture or a second reaction mixture under conditions suitable to extend said cDNA molecule: (i) said cDNA molecule; (ii) a forward primer configured to hybridize to said first synthetic primer hybridization sequence; and (iii) a reverse primer. In some embodiments, said reverse primer is configured to hybridize to said second synthetic primer hybridization sequence. In some embodiments, said reaction mixture further comprises a cell or cellular RNA. In some embodiments, said cell or cellular RNA comprises targeted RNA molecules complementary to said RNA molecule. In some embodiments, said reaction mixture comprises a reverse transcriptase. In some embodiments, said reverse transcriptase comprises Maxima™ H Minus reverse transcriptase, SuperScript™ II reverse transcriptase, Superscript™ III reverse transcriptase, SuperScript™ IV reverse transcriptase, Template Switching RT enzyme mix, SMARTscribe reverse transcriptase, PrimeScript™ RT, or any combination thereof. In some embodiments, said first synthetic primer hybridization sequence comprises a sequence according to 5′-GGACAGAAACATAACATCAGAGT-3′ (SEQ ID NO: 5) or a sequence having at least 90%, 91%, 92%, 93%, 94%, 95%, 96%, 97%, 98%, 99%, or 100% sequence identity thereto. In some embodiments, said second synthetic primer hybridization sequence comprises a sequence according to 5′-CAGTGGTATCAACGCAG-3′ (SEQ ID NO: 6).

The TSO can comprise a variety of structures suitable for template switching with a reverse transcriptase. In some cases, the TSO provides an exogenous template configured for elongation of said RNA molecule. In some embodiments, the exogenous template of said TSO comprises a reverse complement of a second synthetic primer hybridization sequence. In some embodiments, said exogenous template is exogenous to said cell, said tissue, or said organism from which said RNA molecule was obtained or derived. In some embodiments, said complement of said exogenous template is not configured to hybridize to a cellular RNA from said cell, said tissue, or said organism from which said RNA molecule was obtained or derived. In some embodiments, said exogenous template comprises a sequence exogenous to said RNA molecule. In some embodiments, a complement of said exogenous template is not configured to bind to said RNA molecule. In some embodiments, said TSO comprises a 3′ end comprising rGrG. In some embodiments, said TSO comprises a 3′ terminal locked nucleic acid guanine (+G). In some embodiments, said TSO comprises a 3′ terminal sequence of rGrG+G. In some embodiments, said 5′ end sequence of said TSO comprises a reverse complement of a second synthetic primer hybridization sequence. In some embodiments, said TSO comprises a 5′ end comprising at least one isoguanine (iG) or isocytosine (iC) residue. In some embodiments, said 5′ end comprising at least one isoguanine or isocytosine residue comprises iGiC. In some embodiments, the exogenous template is at most about 50, 45, 40, 35, 30, 29, 28, 27, 26, 25, 24, 23, 22, 21, 20, 19, 18, 17. 16, 15, 14, 13, 12, 11, 10, 9, 8, 7, 6, or 5 nucleotides in length, or any range between these values. In some embodiments, the exogenous template is at least about 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, or 45 nucleotides in length, or any range between these values. In some embodiments, the exogenous template is about 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, or 45 nucleotides in length to about 50, 45, 40, 35, 30, 29, 28, 27, 26, 25, 24, 23, 22, 21,20, 19, 18, 17. 16, 15, 14, 13, 12, 11, 10, 9, 8, 7, 6, or 5 nucleotides in length. In some embodiments, said exogenous template comprises fewer than or equal to 48, 47, 45, 40, 35, 30, 29, 28, 27, 26, 25, 24, 23, 22, 21, 20, 19, 18, 17. 16, 15, 14, 13, 12, 11, 10, 9, 8, 7, 6, 5, 4, or 3 consecutive bases of said RNA molecule or a complement thereof.

The stem-loop primer can comprise a variety of features suitable for forming a stable stem-loop under experimental conditions. In some embodiments, the stem-loop primer comprises: a 5′ hemiprobe segment configured to hybridize to a first end region of said RNA molecule; a stem-loop; and a 3′ hemiprobe segment configured to hybridize to a second end region of said RNA molecule. In some embodiments, said first end region comprises an at least partially complementary first terminus. In some embodiments, said second end region comprises an at least partially complementary second terminus. In some embodiments, said stem-loop segment comprises a first synthetic primer hybridization sequence. In some embodiments, said 5′ hemiprobe comprises at least about 5 to at most about 20 nucleotides. In some embodiments, said 5′ hemiprobe comprises at least about 5, 6, 7, 8, 9, 10, 11, 12, 13, 14, 15, 16, 17, 18, or 19 nucleotides. In some embodiments, said 5′ hemiprobe comprises at most about 20, 19, 18, 17, 16, 15, 14, 13, 12, 11, 10, 9, 8, 7, or 6 nucleotides. In some embodiments said 5′ hemiprobe comprises at least about 5, 6, 7, 8, 9, 10, 11, 12, 13, 14, 15, 16, 17, 18, or 19 nucleotides to at most about 20, 19, 18, 17, 16, 15, 14, 13, 12, 11, 10, 9, 8, 7, or 6 nucleotides, or any range between these values. In some embodiments, said 3′ hemiprobe comprises at least about 3 to about 10 nucleotides. In some embodiments, said 3′ hemiprobe comprises at least about 3, 4, 5, 6, 7, 8, or 9 nucleotides. In some embodiments, said 3′ hemiprobe comprises at most about 10, 9, 8, 7, 6, 5, or 4 nucleotides. In some embodiments, said 3′ hemiprobe comprises at least about 3, 4, 5, 6, 7, 8, or 9 nucleotides to at most about 10, 9, 8, 7, 6, 5, or 4 nucleotides, or any range between these values. In some embodiments, said stem-loop comprises at least about 20 to about 80 nucleotides. In some embodiments, said stem-loop comprises at least about 20, 21, 22, 23, 24, 25, 26, 27, 28, 29, 30, 31, 32, 33, 34, 35 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, or 79 nucleotides. In some embodiments, said stem-loop comprises at most about 21, 22, 23, 24, 25, 26, 27, 28, 29, 30, 31, 32, 33, 34, 35 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides. In some embodiments, said stem-loop comprises at least about 20, 21, 22, 23, 24, 25, 26, 27, 28, 29, 30, 31, 32, 33, 34, 35 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, or 79 nucleotides to at most about 21, 22, 23, 24, 25, 26, 27, 28, 29, 30, 31, 32, 33, 34, 35 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotide. In some embodiments, a Tm of said stem-loop is from about 40° C. to about 80° C. In some embodiments, a Tm of said stem-loop is at least about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, or 79° C. In some embodiments, a Tm of said stem-loop is at most about 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80° C. In some embodiments, a Tm of said stem-loop is from at least about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, or 79° C. to at most about 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80° C. . . . In some embodiments, a Tm of said stem-loop is from about 60° C. to about 72° C. In some embodiments, a Tm of said stem-loop is at least about 60, 61, 62, 63, 64, 65, 66, 67, 68, 69, 70, or 71° C. In some embodiments, a Tm of said stem-loop is at most about 61, 62, 63, 64, 65, 66, 67, 68, 69, 70, 71, or 72° C. In some embodiments, a Tm of said stem-loop is from at least about 60, 61, 62, 63, 64, 65, 66, 67, 68, 69, 70, or 71° C. to at most about 61, 62, 63, 64, 65, 66, 67, 68, 69, 70, 71, or 72° C.

In some aspects, the present disclosure provides for a method for processing an RNA molecule, comprising: (a) providing in a reaction mixture under conditions suitable for extending said RNA molecule: (i) said RNA molecule, which comprises a synthetic oligonucleotide or a synthetic linkage between two nucleotides of said RNA molecule; and (ii) a stem-loop primer comprising a first synthetic primer hybridization sequence; and (b) extending said stem-loop primer according to said RNA molecule under conditions suitable to incorporate a 3′ region comprising a second synthetic primer hybridization sequence, thereby generating a cDNA molecule from said RNA molecule and said stem-loop primer. In some embodiments, the method further comprises detecting said cDNA molecule via said first synthetic primer hybridization sequence and said second synthetic primer hybridization sequence. In some embodiments, extending said stem-loop primer according to said RNA molecule under conditions suitable to incorporate a 3′ region comprising a second synthetic primer hybridization sequence comprises providing in a reaction mixture under conditions suitable for extending said RNA molecule: (iii) a template switch oligonucleotide (TSO). In some embodiments, said cDNA molecule is detected at least in part via a synthetic primer hybridization sequence contained within said TSO or said stem-loop primer. In some embodiments, said detecting further comprises quantitative PCR (qPCR), digital PCR (dPCR), sequencing, or array hybridization. In some embodiments, said detecting further comprises contacting said cDNA with a probe oligo configured to hybridize to an internal region of said cDNA. In some embodiments, said detecting further comprises contacting said cDNA with a probe oligo configured to hybridize to an internal region of said cDNA molecule complementary to said RNA molecule. In some embodiments, said probe oligo is configured to hybridize a region of a sequence complementary to said RNA molecule within said cDNA molecule. In some embodiments, said probe oligo is a hybridization probe or hydrolysis probe. In some embodiments, said probe oligo is configured to hybridize to a region of said stem-loop within said cDNA molecule. In some embodiments, said detecting further comprises combining in said reaction mixture or a second reaction mixture under conditions suitable to extend said cDNA molecule: (i) said cDNA molecule; (ii) a forward primer configured to hybridize to said first synthetic primer hybridization sequence; and (iii) a reverse primer. In some embodiments, said reverse primer is configured to hybridize to said second synthetic primer hybridization sequence. In some embodiments, said reaction mixture further comprises a cell or cellular RNA. In some embodiments, said cell or cellular RNA comprises targeted RNA molecules complementary to said RNA molecule. In some embodiments, said reaction mixture comprises a reverse transcriptase. In some embodiments, said reverse transcriptase comprises Maxima™ H Minus reverse transcriptase, SuperScript™ II reverse transcriptase, Superscript™ III reverse transcriptase, SuperScript™ IV reverse transcriptase, Template Switching RT enzyme mix, SMARTscribe reverse transcriptase, PrimeScript™ RT, or any combination thereof. In some embodiments, said first synthetic primer hybridization sequence comprises a sequence according to 5′-GGACAGAAACATAACATCAGAGT-3′ (SEQ ID NO: 5) or a sequence having at least 90%, 91%, 92%, 93%, 94%, 95%, 96%, 97%, 98%, 99%, or 100% sequence identity thereto. In some embodiments, said second synthetic primer hybridization sequence comprises a sequence according to 5′-CAGTGGTATCAACGCAG-3′ (SEQ ID NO: 6). In some embodiments, the method further comprises detecting said cDNA molecule via said first synthetic primer hybridization sequence and said second synthetic primer hybridization sequence. In some embodiments, said second synthetic primer hybridization sequence is exogenous to a cell, tissue, or organism from which said RNA molecule is obtained or derived. In some embodiments, a complement of said second synthetic primer hybridization sequence is not configured to bind to said RNA molecule. In some embodiments, said RNA molecule is obtained from a cell, a tissue, or an organism. In some embodiments, said second synthetic primer hybridization sequence is exogenous to said cell, said tissue, or said organism. In some embodiments, said a complement of said second synthetic primer hybridization sequence is not configured to hybridize to a cellular RNA from said cell, said tissue, or said organism. In some embodiments, said reaction mixture further comprises a cell or cellular RNA. In some embodiments, said cell or cellular RNA comprises targeted RNA molecules complementary to said RNA molecule.

The TSO can comprise a variety of structures suitable for template switching with a reverse transcriptase. In some cases, the TSO provides an exogenous template configured for elongation of said RNA molecule. In some embodiments, the exogenous template of said TSO comprises a reverse complement of a second synthetic primer hybridization sequence. In some embodiments, said exogenous template is exogenous to said cell, said tissue, or said organism from which said RNA molecule was obtained or derived. In some embodiments, said complement of said exogenous template is not configured to hybridize to a cellular RNA from said cell, said tissue, or said organism from which said RNA molecule was obtained or derived. In some embodiments, said exogenous template comprises a sequence exogenous to said RNA molecule. In some embodiments, a complement of said exogenous template is not configured to bind to said RNA molecule. In some embodiments, said TSO comprises a 3′ end comprising rGrG. In some embodiments, said TSO comprises a 3′ terminal locked nucleic acid guanine (+G). In some embodiments, said TSO comprises a 3′ terminal sequence of rGrG+G. In some embodiments, said 5′ end sequence of said TSO comprises a reverse complement of a second synthetic primer hybridization sequence. In some embodiments, said TSO comprises a 5′ end comprising at least one isoguanine (iG) or isocytosine (iC) residue. In some embodiments, said 5′ end comprising at least one isoguanine or isocytosine residue comprises iGiC. In some embodiments, the exogenous template is at most about 50, 45, 40, 35, 30, 29, 28, 27, 26, 25, 24, 23, 22, 21,20, 19, 18, 17. 16, 15, 14, 13, 12, 11, 10, 9, 8, 7, 6, or 5 nucleotides in length, or any range between these values. In some embodiments, the exogenous template is at least about 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, or 45 nucleotides in length, or any range between these values. In some embodiments, the exogenous template is about 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, or 45 nucleotides in length to about 50, 45, 40, 35, 30, 29, 28, 27, 26, 25, 24, 23, 22, 21,20, 19, 18, 17. 16, 15, 14, 13, 12, 11, 10, 9, 8, 7, 6, or 5 nucleotides in length. In some embodiments, said exogenous template comprises fewer than or equal to 48, 47, 45, 40, 35, 30, 29, 28, 27, 26, 25, 24, 23, 22, 21, 20, 19, 18, 17. 16, 15, 14, 13, 12, 11, 10, 9, 8, 7, 6, 5, 4, or 3 consecutive bases of said RNA molecule or a complement thereof.

The stem-loop primer can comprise a variety of features suitable for forming a stable stem-loop under experimental conditions. In some embodiments, the stem-loop primer comprises: a 5′ hemiprobe segment configured to hybridize to a first end region of said RNA molecule; a stem-loop; and a 3′ hemiprobe segment configured to hybridize to a second end region of said RNA molecule. In some embodiments, said first end region comprises an at least partially complementary first terminus. In some embodiments, said second end region comprises an at least partially complementary second terminus. In some embodiments, said stem-loop segment comprises a first synthetic primer hybridization sequence. In some embodiments, said 5′ hemiprobe comprises at least about 5 to at most about 20 nucleotides. In some embodiments, said 5′ hemiprobe comprises at least about 5, 6, 7, 8, 9, 10, 11, 12, 13, 14, 15, 16, 17, 18, or 19 nucleotides. In some embodiments, said 5′ hemiprobe comprises at most about 20, 19, 18, 17, 16, 15, 14, 13, 12, 11, 10, 9, 8, 7, or 6 nucleotides. In some embodiments said 5′ hemiprobe comprises at least about 5, 6, 7, 8, 9, 10, 11, 12, 13, 14, 15, 16, 17, 18, or 19 nucleotides to at most about 20, 19, 18, 17, 16, 15, 14, 13, 12, 11, 10, 9, 8, 7, or 6 nucleotides, or any range between these values. In some embodiments, said 3′ hemiprobe comprises at least about 3 to about 10 nucleotides. In some embodiments, said 3′ hemiprobe comprises at least about 3, 4, 5, 6, 7, 8, or 9 nucleotides. In some embodiments, said 3′ hemiprobe comprises at most about 10, 9, 8, 7, 6, 5, or 4 nucleotides. In some embodiments, said 3′ hemiprobe comprises at least about 3, 4, 5, 6, 7, 8, or 9 nucleotides to at most about 10, 9, 8, 7, 6, 5, or 4 nucleotides, or any range between these values. In some embodiments, said stem-loop comprises at least about 20 to about 80 nucleotides. In some embodiments, said stem-loop comprises at least about 20, 21, 22, 23, 24, 25, 26, 27, 28, 29, 30, 31, 32, 33, 34, 35 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, or 79 nucleotides. In some embodiments, said stem-loop comprises at most about 21, 22, 23, 24, 25, 26, 27, 28, 29, 30, 31, 32, 33, 34, 35 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides. In some embodiments, said stem-loop comprises at least about 20, 21, 22, 23, 24, 25, 26, 27, 28, 29, 30, 31, 32, 33, 34, 35 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, or 79 nucleotides to at most about 21, 22, 23, 24, 25, 26, 27, 28, 29, 30, 31, 32, 33, 34, 35 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotide. In some embodiments, a Tm of said stem-loop is from about 40° C. to about 80° C. In some embodiments, a Tm of said stem-loop is at least about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, or 79° C. In some embodiments, a Tm of said stem-loop is at most about 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80° C. In some embodiments, a Tm of said stem-loop is from at least about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, or 79° C. to at most about 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80° C. . . . In some embodiments, a Tm of said stem-loop is from about 60° C. to about 72° C. In some embodiments, a Tm of said stem-loop is at least about 60, 61, 62, 63, 64, 65, 66, 67, 68, 69, 70, or 71° C. In some embodiments, a Tm of said stem-loop is at most about 61, 62, 63, 64, 65, 66, 67, 68, 69, 70, 71, or 72° C. In some embodiments, a Tm of said stem-loop is from at least about 60, 61, 62, 63, 64, 65, 66, 67, 68, 69, 70, or 71° C. to at most about 61, 62, 63, 64, 65, 66, 67, 68, 69, 70, 71, or 72° C. . . .

In some aspects, the present disclosure provides for a kit for detecting an RNA molecule, comprising: (a) a stem-loop primer; (b) a template switch oligonucleotide (TSO) providing an exogenous template configured for elongation of said RNA molecule; and (c) instructions for detecting said RNA molecule using said stem-loop primer and said template switch oligonucleotide. In some embodiments, the stem-loop primer further comprises a 5′ hemiprobe segment configured to hybridize to a complementary first terminus of said RNA molecule; a stem-loop segment; and a 3′ hemiprobe segment configured to hybridize to a complementary second terminus of said RNA molecule In some embodiments, the kit further comprises an RNAse inhibitor solution. In some embodiments, the kit further comprises a reverse transcriptase. In some embodiments, said reverse transcriptase comprises Maxima™ H Minus reverse transcriptase, SuperScript™ II reverse transcriptase, Superscript™ III reverse transcriptase, SuperScript™ IV reverse transcriptase, Template Switching RT enzyme mix, SMARTscribe reverse transcriptase, PrimeScript™ RT, or any combination thereof. In some embodiments, the kit further comprises a fluorophore-linked oligonucleotide. In some embodiments, the fluorophore-linked oligonucleotide further comprises a quencher.

The TSO can comprise a variety of structures suitable for template switching with a reverse transcriptase. In some cases, the TSO provides an exogenous template configured for elongation of said RNA molecule. In some embodiments, the exogenous template of said TSO comprises a reverse complement of a second synthetic primer hybridization sequence. In some embodiments, said exogenous template is exogenous to said cell, said tissue, or said organism from which said RNA molecule was obtained or derived. In some embodiments, said complement of said exogenous template is not configured to hybridize to a cellular RNA from said cell, said tissue, or said organism from which said RNA molecule was obtained or derived. In some embodiments, said exogenous template comprises a sequence exogenous to said RNA molecule. In some embodiments, a complement of said exogenous template is not configured to bind to said RNA molecule. In some embodiments, said TSO comprises a 3′ end comprising rGrG. In some embodiments, said TSO comprises a 3′ terminal locked nucleic acid guanine (+G). In some embodiments, said TSO comprises a 3′ terminal sequence of rGrG+G. In some embodiments, said 5′ end sequence of said TSO comprises a reverse complement of a second synthetic primer hybridization sequence. In some embodiments, said TSO comprises a 5′ end comprising at least one isoguanine (iG) or isocytosine (iC) residue. In some embodiments, said 5′ end comprising at least one isoguanine or isocytosine residue comprises iGiC. In some embodiments, the exogenous template is at most about 50, 45, 40, 35, 30, 29, 28, 27, 26, 25, 24, 23, 22, 21,20, 19, 18, 17. 16, 15, 14, 13, 12, 11, 10, 9, 8, 7, 6, or 5 nucleotides in length, or any range between these values. In some embodiments, the exogenous template is at least about 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, or 45 nucleotides in length, or any range between these values. In some embodiments, the exogenous template is about 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, or 45 nucleotides in length to about 50, 45, 40, 35, 30, 29, 28, 27, 26, 25, 24, 23, 22, 21,20, 19, 18, 17. 16, 15, 14, 13, 12, 11, 10, 9, 8, 7, 6, or 5 nucleotides in length. In some embodiments, said exogenous template comprises fewer than or equal to 48, 47, 45, 40, 35, 30, 29, 28, 27, 26, 25, 24, 23, 22, 21, 20, 19, 18, 17. 16, 15, 14, 13, 12, 11, 10, 9, 8, 7, 6, 5, 4, or 3 consecutive bases of said RNA molecule or a complement thereof.

The stem-loop primer can comprise a variety of features suitable for forming a stable stem-loop under experimental conditions. In some embodiments, the stem-loop primer comprises: a 5′ hemiprobe segment configured to hybridize to a first end region of said RNA molecule; a stem-loop; and a 3′ hemiprobe segment configured to hybridize to a second end region of said RNA molecule. In some embodiments, said first end region comprises an at least partially complementary first terminus. In some embodiments, said second end region comprises an at least partially complementary second terminus. In some embodiments, said stem-loop segment comprises a first synthetic primer hybridization sequence. In some embodiments, said 5′ hemiprobe comprises at least about 5 to at most about 20 nucleotides. In some embodiments, said 5′ hemiprobe comprises at least about 5, 6, 7, 8, 9, 10, 11, 12, 13, 14, 15, 16, 17, 18, or 19 nucleotides. In some embodiments, said 5′ hemiprobe comprises at most about 20, 19, 18, 17, 16, 15, 14, 13, 12, 11, 10, 9, 8, 7, or 6 nucleotides. In some embodiments said 5′ hemiprobe comprises at least about 5, 6, 7, 8, 9, 10, 11, 12, 13, 14, 15, 16, 17, 18, or 19 nucleotides to at most about 20, 19, 18, 17, 16, 15, 14, 13, 12, 11, 10, 9, 8, 7, or 6 nucleotides, or any range between these values. In some embodiments, said 3′ hemiprobe comprises at least about 3 to about 10 nucleotides. In some embodiments, said 3′ hemiprobe comprises at least about 3, 4, 5, 6, 7, 8, or 9 nucleotides. In some embodiments, said 3′ hemiprobe comprises at most about 10, 9, 8, 7, 6, 5, or 4 nucleotides. In some embodiments, said 3′ hemiprobe comprises at least about 3, 4, 5, 6, 7, 8, or 9 nucleotides to at most about 10, 9, 8, 7, 6, 5, or 4 nucleotides, or any range between these values. In some embodiments, said stem-loop comprises at least about 20 to about 80 nucleotides. In some embodiments, said stem-loop comprises at least about 20, 21, 22, 23, 24, 25, 26, 27, 28, 29, 30, 31, 32, 33, 34, 35 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, or 79 nucleotides. In some embodiments, said stem-loop comprises at most about 21, 22, 23, 24, 25, 26, 27, 28, 29, 30, 31, 32, 33, 34, 35 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides. In some embodiments, said stem-loop comprises at least about 20, 21, 22, 23, 24, 25, 26, 27, 28, 29, 30, 31, 32, 33, 34, 35 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, or 79 nucleotides to at most about 21, 22, 23, 24, 25, 26, 27, 28, 29, 30, 31, 32, 33, 34, 35 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotide. In some embodiments, a Tm of said stem-loop is from about 40° C. to about 80° C. In some embodiments, a Tm of said stem-loop is at least about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, or 79° C. In some embodiments, a Tm of said stem-loop is at most about 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80° C. In some embodiments, a Tm of said stem-loop is from at least about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, or 79° C. to at most about 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80° C. . . . In some embodiments, a Tm of said stem-loop is from about 60° C. to about 72° C. In some embodiments, a Tm of said stem-loop is at least about 60, 61, 62, 63, 64, 65, 66, 67, 68, 69, 70, or 71° C. In some embodiments, a Tm of said stem-loop is at most about 61, 62, 63, 64, 65, 66, 67, 68, 69, 70, 71, or 72° C. In some embodiments, a Tm of said stem-loop is from at least about 60, 61, 62, 63, 64, 65, 66, 67, 68, 69, 70, or 71° C. to at most about 61, 62, 63, 64, 65, 66, 67, 68, 69, 70, 71, or 72° C.

In some aspects, the present disclosure provides for (a) an RNA molecule; and (b) a stem-loop primer; and (c) a template switch oligonucleotide (TSO) providing an exogenous template configured for 5′ end elongation of said RNA molecule. In some embodiments, said mixture further comprises an RNAse inhibitor. In some embodiments, said mixture further comprises a reverse transcriptase. In some embodiments, said reverse transcriptase comprises Maxima™ H Minus reverse transcriptase, SuperScript™ II reverse transcriptase, Superscript™ III reverse transcriptase, SuperScript™ IV reverse transcriptase, Template Switching RT enzyme mix, SMARTscribe reverse transcriptase, PrimeScript™ RT, or any combination thereof. In some embodiments, the mixture further comprises a fluorophore-linked oligonucleotide. In some embodiments, the fluorophore-linked oligonucleotide further comprises a quencher. In some cases, the RNA molecule is a short RNA molecule, a short interfering RNA (siRNA), a microRNA (miRNA), a transfer RNA, a ribosomal RNA, a short-hairpin RNA (shRNA), or a small nucleolar RNA. In some cases, said RNA molecule is a naturally-occurring RNA molecule. In some cases, said RNA molecule is obtained from a sample from cell, a tissue, or an organism. In some embodiments, said RNA molecule comprises a synthetic sugar-modified oligonucleotide or a synthetic linkage between two nucleotides of said RNA molecule. In some embodiments, said RNA molecule comprises a phosphorothioate linkage, a 2′-fluororibonucleotide, a 2′-O-methylribonucleotide, or a 2′-O-methoxyethylribonucleotide.

The TSO can comprise a variety of structures suitable for template switching with a reverse transcriptase. In some cases, the TSO provides an exogenous template configured for elongation of said RNA molecule. In some embodiments, the exogenous template of said TSO comprises a reverse complement of a second synthetic primer hybridization sequence. In some embodiments, said exogenous template is exogenous to said cell, said tissue, or said organism from which said RNA molecule was obtained or derived. In some embodiments, said complement of said exogenous template is not configured to hybridize to a cellular RNA from said cell, said tissue, or said organism from which said RNA molecule was obtained or derived. In some embodiments, said exogenous template comprises a sequence exogenous to said RNA molecule. In some embodiments, a complement of said exogenous template is not configured to bind to said RNA molecule. In some embodiments, said TSO comprises a 3′ end comprising rGrG. In some embodiments, said TSO comprises a 3′ terminal locked nucleic acid guanine (+G). In some embodiments, said TSO comprises a 3′ terminal sequence of rGrG+G. In some embodiments, said 5′ end sequence of said TSO comprises a reverse complement of a second synthetic primer hybridization sequence. In some embodiments, said TSO comprises a 5′ end comprising at least one isoguanine (iG) or isocytosine (iC) residue. In some embodiments, said 5′ end comprising at least one isoguanine or isocytosine residue comprises iGiC. In some embodiments, the exogenous template is at most about 50, 45, 40, 35, 30, 29, 28, 27, 26, 25, 24, 23, 22, 21,20, 19, 18, 17. 16, 15, 14, 13, 12, 11, 10, 9, 8, 7, 6, or 5 nucleotides in length, or any range between these values. In some embodiments, the exogenous template is at least about 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, or 45 nucleotides in length, or any range between these values. In some embodiments, the exogenous template is about 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, or 45 nucleotides in length to about 50, 45, 40, 35, 30, 29, 28, 27, 26, 25, 24, 23, 22, 21,20, 19, 18, 17. 16, 15, 14, 13, 12, 11, 10, 9, 8, 7, 6, or 5 nucleotides in length. In some embodiments, said exogenous template comprises fewer than or equal to 48, 47, 45, 40, 35, 30, 29, 28, 27, 26, 25, 24, 23, 22, 21, 20, 19, 18, 17. 16, 15, 14, 13, 12, 11, 10, 9, 8, 7, 6, 5, 4, or 3 consecutive bases of said RNA molecule or a complement thereof.

The stem-loop primer can comprise a variety of features suitable for forming a stable stem-loop under experimental conditions. In some embodiments, the stem-loop primer comprises: a 5′ hemiprobe segment configured to hybridize to a first end region of said RNA molecule; a stem-loop; and a 3′ hemiprobe segment configured to hybridize to a second end region of said RNA molecule. In some embodiments, said first end region comprises an at least partially complementary first terminus. In some embodiments, said second end region comprises an at least partially complementary second terminus. In some embodiments, said stem-loop segment comprises a first synthetic primer hybridization sequence. In some embodiments, said 5′ hemiprobe comprises at least about 5 to at most about 20 nucleotides. In some embodiments, said 5′ hemiprobe comprises at least about 5, 6, 7, 8, 9, 10, 11, 12, 13, 14, 15, 16, 17, 18, or 19 nucleotides. In some embodiments, said 5′ hemiprobe comprises at most about 20, 19, 18, 17, 16, 15, 14, 13, 12, 11, 10, 9, 8, 7, or 6 nucleotides. In some embodiments said 5′ hemiprobe comprises at least about 5, 6, 7, 8, 9, 10, 11, 12, 13, 14, 15, 16, 17, 18, or 19 nucleotides to at most about 20, 19, 18, 17, 16, 15, 14, 13, 12, 11, 10, 9, 8, 7, or 6 nucleotides, or any range between these values. In some embodiments, said 3′ hemiprobe comprises at least about 3 to about 10 nucleotides. In some embodiments, said 3′ hemiprobe comprises at least about 3, 4, 5, 6, 7, 8, or 9 nucleotides. In some embodiments, said 3′ hemiprobe comprises at most about 10, 9, 8, 7, 6, 5, or 4 nucleotides. In some embodiments, said 3′ hemiprobe comprises at least about 3, 4, 5, 6, 7, 8, or 9 nucleotides to at most about 10, 9, 8, 7, 6, 5, or 4 nucleotides, or any range between these values. In some embodiments, said stem-loop comprises at least about 20 to about 80 nucleotides. In some embodiments, said stem-loop comprises at least about 20, 21, 22, 23, 24, 25, 26, 27, 28, 29, 30, 31, 32, 33, 34, 35 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, or 79 nucleotides. In some embodiments, said stem-loop comprises at most about 21, 22, 23, 24, 25, 26, 27, 28, 29, 30, 31, 32, 33, 34, 35 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides. In some embodiments, said stem-loop comprises at least about 20, 21, 22, 23, 24, 25, 26, 27, 28, 29, 30, 31, 32, 33, 34, 35 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, or 79 nucleotides to at most about 21, 22, 23, 24, 25, 26, 27, 28, 29, 30, 31, 32, 33, 34, 35 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotide. In some embodiments, a Tm of said stem-loop is from about 40° C. to about 80° C. In some embodiments, a Tm of said stem-loop is at least about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, or 79° C. In some embodiments, a Tm of said stem-loop is at most about 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80° C. In some embodiments, a Tm of said stem-loop is from at least about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, or 79° C. to at most about 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80° C. In some embodiments, a Tm of said stem-loop is from about 60° C. to about 72° C. In some embodiments, a Tm of said stem-loop is at least about 60, 61, 62, 63, 64, 65, 66, 67, 68, 69, 70, or 71° C. In some embodiments, a Tm of said stem-loop is at most about 61, 62, 63, 64, 65, 66, 67, 68, 69, 70, 71, or 72° C. In some embodiments, a Tm of said stem-loop is from at least about 60, 61, 62, 63, 64, 65, 66, 67, 68, 69, 70, or 71° C. to at most about 61, 62, 63, 64, 65, 66, 67, 68, 69, 70, 71, or 72° C.

In some aspects, the present disclosure provides for a nucleic acid comprising a sequence having at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to any of the sequences described in Table A, or a reverse complement thereof.

TABLE A
Sequences of nucleic acids utilized with methods according to the disclosure.

| SEQ ID | | |
|---|---|---|
| NO: | DESCRIPTION | SEQUENCE |
| 1 | miR-1 | UAUUUUCUGCAUUCGCCCUUGC |
| | (1st strand) | |
| | (Example 1) | |
| 2 | miR-1 | AAGGAGCGAUUUGGAGAAAAUAAA |
| | (2^{nd} strand) | |
| | (Example 1) | |
| 3 | 2T primer | CAGAAACATAACATCAGAGTTGTTACAGTGAGTAACAA |
| | (Example 1) | TATGGTTGAAGGGC |
| 4 | TSO | AAGCAGTGGTATCAACGCAGAGTACATrGrG+G |
| | (Example 1) | |
| 5 | Fw primer | GGACAGAAACATAACATCAGAGT |
| | (Example 1) | |
| 6 | Rev primer | CAGTGGTATCAACGCAG |
| | (Example 1) | |
| 7 | Probe | TGGTTGAAGGGCGAATGCAGAAAATA |
| | (Example 1) | |

Legend:
rG; riboguanosine. +G; LNA guanosine; rN: ribonucleotide. N: nucleotide A, U, C or G

### EXAMPLES

### Example 1.—Optimization of Reverse Transcriptase Conditions for Short RNA (e.g. sRNA, miRNA) Detection Assay

Eight different reaction temperatures were tested at the reverse transcription (RT)/template switching (TS) step for the reaction depicted in FIG. 1. Two reverse transcriptases (RTases) were also tested: SuperScript II (ThermoFisher 18064014) and Maxima H Minus (ThermoFisher EP0752). The target analyte was a synthetic single-stranded, 22 nt long, microRNA (miRNA). The miRNA was purchased freeze-dried from IDT, resuspended in Tris-EDTA (TE) buffer and further diluted to 1E+8 cp/μL in water. All oligo sequences are presented in Table 1. Negative control reactions contained nuclease-free water instead of siRNA.

TABLE 1
Sequences of oligos used in Example 1.

| Description | Sequence |
|---|---|
| miR-1 | UAUUUUCUGCAUUCGCCCUUGC (SEQ ID NO: 1) |
| (1st strand) | |
| miR-1 | AAGGAGCGAUUUGGAGAAAAUAAA (SEQ ID NO: 2) |
| (2^{nd} strand) | |
| (Example 1) | |
| 2T primer | CAGAAACATAACATCAGAGTTGTTACAGTGAGTAACAATATGGTTGAAGGGC |
| | (SEQ ID NO: 3) |
| TSO | AAGCAGTGGTATCAACGCAGAGTACATrGrG+G (SEQ ID NO: 4) |
| Fw primer | GGACAGAAACATAACATCAGAGT (SEQ ID NO: 5) |
| Rev primer | CAGTGGTATCAACGCAG (SEQ ID NO: 6) |
| Probe | TGGTTGAAGGGCGAATGCAGAAAATA (SEQ ID NO: 7) |

rG; riboguanosine. +G; LNA guanosine; rN: ribonucleotide. N: nucleotide A, U, C or G

### Reaction Assembly and Execution

For the reaction using Maxima reverse transcriptase, each 10 μL RT reaction comprised 1.5× RT buffer (ThermoFisher), 0.6 μM template switch oligo (TSO), 1.5 mM dNTP mix, 20 nM 2-tail (2T) primer, 15 U RiboLock RNase inhibitor (ThermoFisher EO0381), 50 U of Maxima H Minus RTase and 5E+8 copies (cp) miRNA.

For the reaction using SuperScriptII reverse transcriptase, each 10 μL RT reaction comprised 1.5× first-stand buffer (ThermoFisher), 7.5 mM DTT, 0.6 μM, 1.5 mM dNTP mix, 20 nM 2T primer, 15 U RiboLock RNase inhibitor (ThermoFisher EO0381), 50 U of SuperScritpt II RTase and 5E+8 cp siRNA.

The reverse transcriptase (RT) reactions were incubated in a thermal cycler with temperature gradient function for 90 min at the test temperature (FIG. 2A: 37° C., 40° C., 48° C. and 55° C., FIG. 2b: 25° C., 32° C., 37.5° C. and 42° C.). A period of enzyme inactivation followed (70° C. for 15 min). The resulting cDNA was diluted 5× before usage in qPCR.

Quantitative PCR (qPCR) Assembly and Execution

For the qPCR reaction on the reaction products generated above, each 10 μL reaction was composed of 1×TATAA probe GrandMaster mix (TA02-62), 400 nM Fw and Rev primers, 200 nM probe and 2 μL diluted cDNA template. qPCR non-template control (NTC) reactions: water added instead of cDNA. Cycling conditions: 1 minute denaturation (95° C.), followed by 45 cycles comprising 5 seconds denaturation (95° C.) and 30 seconds extension (57° C.).

FIGS. 2A and 2B depict results of the experiment (table shown is also reproduced as Table 2 below). Shown are tables of reverse transcriptase step temp (“RT temp”) and quantification cycle (Cq) alongside a qPCR trace of RFU versus cycle. RT reactions were run at 37° C., 40° C., 48° C. and 55° C. for FIG. 2A, or 25° C., 32° C., 37.5° C. and 42° C. for FIG. 2B. Two RTases were tested: SuperScript II and Maxima H Minus. Reactions were run with 0.6 μM TSO (“+TSO”). RT negative controls contained either no TSO (“-TSO”), no 2T primer (“-2T”) or no miRNA (“-miRNA”). qPCR NTC: water was added instead of RT product to the qPCR reaction. The results show that the method of FIG. 1A accommodates multiple different reverse transcriptases.

TABLE 2
Table of reverse transcriptase step temp (“RT temp”) and
quantification cycle (Cq) for the experiment of Example 1.

| RTase | Oligos added | RT temp | Cq |
|---|---|---|---|
| SuperScript II | +miRNA +TSO +2T | 55° C. | N/A |
| | | 48° C. | N/A |
| | | 40° C. | 37.06 |
| | | 37° C. | N/A |
| | +miRNA −TSO +2T | 55° C. | N/A |
| | | 48° C. | N/A |
| | | 40° C. | N/A |
| | | 37° C. | N/A |
| | +miRNA +TSO −2T | 55° C. | N/A |
| | | 48° C. | N/A |
| | | 40° C. | N/A |
| | | 37° C. | N/A |
| | −miRNA +TSO +2T | 55° C. | N/A |
| | | 48° C. | N/A |
| | | 40° C. | N/A |
| | | 37° C. | N/A |
| Maxima H Minus | +miRNA +TSO +2T | 55° C. | 33.08 |
| | | 48° C. | 30.42 |
| | | 40° C. | 19.28 |
| | | 37° C. | 18.30 |
| | +miRNA −TSO +2T | 55° C. | N/A |
| | | 48° C. | N/A |
| | | 40° C. | N/A |
| | | 37° C. | N/A |
| | +miRNA +TSO −2T | 55° C. | N/A |
| | | 48° C. | N/A |
| | | 40° C. | N/A |
| | | 37° C. | N/A |
| | −miRNA +TSO +2T | 55° C. | N/A |
| | | 48° C. | N/A |
| | | 40° C. | N/A |
| | | 37° C. | N/A |

| | |
|---|---|
| qPCR NTC | N/A |
| qPCR NTC | N/A |

### Example 2.—Evaluation of Sensitivity of Short RNA (e.g. siRNA) Detection Assay

Assay sensitivity was evaluated by making a ten-fold dilution series of siRNA_1 in TE-LPA buffer (20 ng/μl GenElute-LPA (Sigma-Aldrich 56575) in TE). The starting concentration was 1E+9 cp/μL and the lowest dilution point was 10 cp/μL. All oligo sequences are presented in Table 1. The siRNA was omitted in the negative RT control.

### Reaction Assembly and Execution

Each 10 μL reaction comprised 1.5× RT buffer (ThermoFisher), 1.5 μM TSO, 1.5 mM dNTP mix, 50 nM 2T primer, 15 U RiboLock RNase inhibitor (ThermoFisher EO0381), 50 U of Maxima H Minus RTase and 1 μL siRNA (1E+9 down to 10 molecules). The RT reactions were incubated in a thermal cycler at 25° C. for 90 min followed by enzyme inactivation at 70° C. for 15 min. The resulting cDNA was diluted 5× before usage in qPCR.

Quantitative PCR (qPCR) Assembly and Execution

Each 10 μL reaction was composed of 1×TATAA probe GrandMaster mix (TA02-62), 400 nM Fw and Rev primers, 200 nM probe and 2 μL diluted cDNA template. qPCR NTC reactions: water added instead of cDNA. The cycling conditions were 1 minute denaturation (95° C.), followed by 45 cycles each comprising 5 seconds denaturation (95° C.) and 30 seconds extension (57° C.).

FIG. 3 depicts results of this experiment (the table is also reproduced as Table 3 below). Shown is a table of the relationship between quantity of RNA (e.g. sRNA, siRNA) and quantitation cycle (Cq) and a graph showing the derived standard curve and associated regression equation. siRNA_1 was diluted ten-fold in TE-LPA buffer to generate an eight-point standard curve. Duplicate samples were set up at the RT step. The siRNA was omitted in the negative RT control (“-siRNA”) whereas the cDNA was omitted in the qPCR control (“qPCR NTC”). The data indicate the assay detected down to 1000 molecules. The data additionally indicate the assay is linear for detecting short RNA (e.g. siRNA) at least between 1000 copies and 1E+9 copies.

TABLE 3
RNA quantity vs. quantitation cycle
for the experiment of Example 2.

| | Starting | log(starting | | | |
|---|---|---|---|---|---|
| Dil. Point | quantity | quantity) | Cq | Cq mean | Cq SD |
| C1 | 100000000 | 8 | 18.52 | 18.38 | 0.189 |
| | | | 18.25 | | |
| C2 | 10000000 | 7 | 21.89 | 21.77 | 0.158 |
| | | | 21.66 | | |
| C3 | 1000000 | 6 | 24.44 | 24.46 | 0.016 |
| | | | 24.47 | | |
| C4 | 100000 | 5 | 26.13 | 26.02 | 0.164 |
| | | | 25.9 | | |
| C5 | 10000 | 4 | 28.91 | 29 | 0.129 |
| | | | 29.09 | | |
| C6 | 1000 | 3 | 33.52 | 33.73 | 0.308 |
| | | | 33.95 | | |
| C7 | 100 | 2 | N/A | 0 | 0 |
| | | | N/A | | |
| C8 | 10 | 1 | N/A | 0 | 0 |
| | | | N/A | | |
| −RNA | | | N/A | 0 | 0 |
| | | | N/A | | |
| qPCR NTC | | | N/A | 0 | 0 |
| | | | N/A | | |

### Example 3.—Evaluation of Sensitivity of Short RNA (e.g. sRNA, siRNA) Detection Assay to Interference by Cellular RNAs

Robustness toward inhibition by cellular RNAs was performed by spiking siRNA_1 into different concentrations of mouse liver RNA. Two experiments were performed: one in undiluted mouse RNA matrix, and one in diluted mouse RNA matrix.

### Experiment 1

For the first experiment, siRNA_1 was spiked into undiluted mouse liver RNA (“matrix”) to final concentration 0.5E+8 cp/μL. For comparison, siRNA_1 was spiked into TE-LPA in parallel, to achieve the same concentration (0.5E+8 cp/μL). All oligo sequences are presented in Table 1. The negative RT control reaction contained water instead of TSO. Two microliter siRNA (in TE-LPA or matrix) was used per RT reaction.

For the RT reaction, each 10 μL reaction comprised 1.5× RT buffer (ThermoFisher), 1.5 μM or 15 nM TSO, 1.5 mM dNTP mix, 50 nM 2T primer, 15 U RiboLock RNase inhibitor (ThermoFisher EO0381), 50 U of Maxima H Minus RTase and 1E+8 cp siRNA (in TE-LPA or a background of matrix RNA). The RT reactions were incubated in a thermal cycler at 25° C. for 90 min followed by enzyme inactivation at 70° C. for 15 min. The resulting cDNA was diluted 5× before qPCR.

For the qPCR reaction each 10 μL reaction contained 1×TATAA probe GrandMaster mix (TA02-62), 400 nM Fw and Rev primers, 200 nM probe and 2 μL diluted cDNA. qPCR NTC reactions had water added instead of cDNA. The cycling conditions were 1 minute denaturation (95° C.), followed by 45 cycles each comprising 5 seconds denaturation (95° C.) and 30 seconds extension (57° C.).

The results are shown in FIG. 4 panel a (also reproduced as Table 4 below). Shown are summary statistics and a qPCR graph of RFU versus cycle. Values are averages of duplicates set up at the RT step. The Cq delay and the flat amplification curves indicate inhibition in undiluted matrix.

### Experiment 2

For the second experiment, the mouse liver matrix was diluted two-fold in TE-LPA in five steps. This created five matrix dilution points to be used at the RT/TS step (1:2, 1:4, 1:8, 1:16 and 1:32). siRNA_1 was spiked into each matrix sample to final concentration 1E+8 cp/μL. For comparison, siRNA_1 was spiked into TE-LPA in parallel (1E+8 cp/μL final concentration). Negative RT control reactions contained TE-LPA instead of siRNA or water instead of TSO. One microliter siRNA (in TE-LPA or matrix) was used per RT reaction. RT reaction conditions and qPCR were otherwise performed as in Experiment 1.

The results are shown in FIG. 4 panel b (also reproduced as Table 5 below). The data confirm the inhibition effect is overcome by diluting the matrix RNA (smallest ΔCq at 1:32 dilution).

TABLE 4
Summary of effect of cellular RNAs without dilution
on Cq of Quantitation assay in Example 3.

| | | | | ΔCq (RNA-TE- |
|---|---|---|---|---|
| Oligos added | Background | Cq mean | Cq SD | LPA) |
| +RNA +TSO | RNA | 25.8 | 0.02 | 7.24 |
| | | 24.69 | 0.19 | |
| | TE-LPA | 18.01 | 0.00 | |
| | | 18 | 0.04 | |
| +RNA +low TSO | RNA | 32.41 | 0.05 | 12.15 |
| | TE-LPA | 20.26 | 0.02 | |
| +RNA −TSO | TE-LPA | 0 | 0 | N/A |

TABLE 5
Summary of effect of cellular RNAs with dilution
on Cq of Quantitation assay in Example 3.

| | Matrix | | |
|---|---|---|---|
| Oligos added | dilution | Cq | ΔCq (RNA-TELPA) |
| +RNA +TSO | TE-LPA | 15.59 | 0 |
| | 1:2 | 16.49 | 0.9 |
| | 1:4 | 16.35 | 0.76 |
| | 1:8 | 16.29 | 0.7 |
| | 1:16 | 16.04 | 0.45 |
| | 1:32 | 15.85 | 0.26 |
| +RNA +low TSO | TE-LPA | 17.46 | 0 |
| | 1:2 | 20.19 | 2.73 |
| | 1:4 | 19.43 | 1.97 |
| | 1:8 | 18.98 | 1.52 |
| | 1:16 | 18.49 | 1.03 |
| | 1:32 | 18.19 | 0.73 |
| +RNA −TSO | TE-LPA | 38.02 | 0 |
| | 1:2 | N/A | N/A |
| | 1:4 | N/A | |
| | 1:8 | N/A | |
| | 1:16 | N/A | |
| | 1:32 | 37.11 | −0.91 |
| −RNA +TSO | 1:2 | N/A | N/A |
| | 1:16 | N/A | |
| | 1:32 | N/A | |

### Example 4.—Evaluation of Sensitivity of Short RNA (e.g. siRNA) Detection Assay to Non-Natural RNA Modifications

The performance of the method of FIG. 1 on short RNAs (e.g. siRNA) with non-natural modifications was evaluated.

Mouse liver RNA matrix was diluted 1:4 and 1:32 in TE-LPA. siRNA 1 and modified versions thereof were spiked into the matrix samples to a final concentration 1E+8 cp/μL. In parallel, the siRNAs were spiked into TE-LPA (1E+8 cp/μL final concentration). Negative RT control reactions contained water instead of TSO or TE-LPA instead of siRNA. All oligo sequences are presented in Table 1 (see Example 1.

Four modified siRNA sequences were tested:
1) siRNA_PS: carries four phosphorothioated RNA residues;
2) siRNA_2′Ome: has two 2′-O-methyl residues;
3) siRNA_2′MOE: has two 2′-O-methoxy-ethyl residues; and
4) siRNA_STC: is fully modified with 2′-O-methyl and 2′-fluoro residues.

Chemical structures of these modifications are depicted in FIG. 5.

### Reaction Assembly and Execution

Each 10 μL reaction consisted of 1.5× RT buffer (ThermoFisher), 1.5 μM or 15 nM TSO, 1.5 mM dNTP mix, 50 nM 2T primer, 15 U RiboLock RNase inhibitor (ThermoFisher EO0381), 50 U of Maxima H Minus RTase and 1E+8 cp siRNA (in TE-LPA or a background of matrix RNA). The RT reactions were incubated in a thermal cycler at 25° C. for 90 min followed by enzyme inactivation at 70° C. for 15 min. The resulting cDNA was diluted 5× before qPCR.

Quantitative PCR (qPCR) Assembly and Execution

Each 10 μL reaction contained 1×TATAA probe GrandMaster mix (TA02-62), 400 nM Fw and Rev primers, 200 nM probe and 2 μL diluted cDNA. qPCR NTC reactions: water added instead of cDNA. The cycling conditions were 1 minute denaturation (95° C.), followed by 45 cycles each consisting of 5 seconds denaturation (95° C.) and 30 seconds extension (57° C.).

FIGS. 6A, 6B, 6C, and 6D (FIGS. 6A, 6B, 6C, and 6D) depict performance of methods according to the disclosure on chemically-modified RNAs (e.g. chemically-modified siRNAs). Displayed are Cq data (Cq data is also reproduced as Tables 6A, 6B, 6C, and 6D below) and amplification plots from qPCR readout of the 2T-TS assay targeting modified siRNAs. FIG. 6A shows results for siRNA with PS (phosphorothioate) modification at positions 19 and 20. FIG. 6B shows results siRNA with 2′-O-me modification at residues 7 and 17. FIG. 6C shows results for siRNA with 2′-O-MOE modification at positions 7 and 18. FIG. 6D shows results for fully modified siRNA (alternating 2′-F and 2′-O-me nucleotides). The data indicate the method of FIG. 1 can detect PS-carrying siRNA without effects on assay sensitivity (a). There is a minor decrease in sensitivity on 2′-O-me siRNA (FIGS. 6B and 6D). As documented previously, RT was less efficient over 2′-O-MOE residues (FIG. 6C).

TABLE 6A
Summary of effect Cq and ΔCq for the different
conditions described in Example 4 on PS-modified sRNA.

| | | | | ΔCq | ΔCq | ΔCq |
|---|---|---|---|---|---|---|
| | | siRNA | | (RNA − TE- | (PS − | (low − high |
| Oligos added | Diluent | type | Cq | LPA) | unmod.) | [TSO]) |
| +siRNA +TSO | TE-LPA | siRNA | 19.51 | 0 | 0 | 0 |
| | Matrix 1:4 | siRNA | 19.08 | −0.43 | 0 | 0 |
| | Matrix 1:32 | siRNA | 19.01 | −0.50 | 0 | 0 |
| | TE-LPA | PS_siRNA | 19.37 | 0 | −0.14 | 0 |
| | Matrix 1:4 | PS_siRNA | 19.25 | −0.12 | 0.17 | 0 |
| | Matrix 1:32 | PS_siRNA | 19.24 | −0.13 | 0.23 | 0 |
| +siRNA +low | TE-LPA | siRNA | 21.48 | 0 | 0 | 1.97 |
| TSO | Matrix 1:4 | siRNA | 22.55 | 1.08 | 0 | 3.48 |
| | Matrix 1:32 | siRNA | 20.20 | −1.27 | 0 | 1.19 |
| | TE-LPA | PS_siRNA | 20.96 | 0 | −0.52 | 1.59 |
| | Matrix 1:4 | PS_siRNA | 21.88 | 0.92 | −0.67 | 2.63 |
| | Matrix 1:32 | PS_siRNA | 21.01 | 0.05 | 0.81 | 1.77 |
| +siRNA −TSO | TE-LPA | siRNA | N/A | N/A | N/A | N/A |
| | Matrix 1:4 | siRNA | N/A | N/A | N/A | N/A |
| | Matrix 1:32 | siRNA | N/A | N/A | N/A | N/A |
| | TE-LPA | PS_siRNA | N/A | N/A | N/A | N/A |
| | Matrix 1:4 | PS_siRNA | N/A | N/A | N/A | N/A |
| | Matrix 1:32 | PS_siRNA | N/A | N/A | N/A | N/A |
| −siRNA +TSO | TE-LPA | siRNA | N/A | N/A | N/A | N/A |
| | Matrix 1:4 | siRNA | N/A | N/A | N/A | N/A |
| | Matrix 1:32 | siRNA | N/A | N/A | N/A | N/A |
| | TE-LPA | PS_siRNA | N/A | N/A | N/A | N/A |
| | Matrix 1:4 | PS_siRNA | N/A | N/A | N/A | N/A |
| | Matrix 1:32 | PS_siRNA | N/A | N/A | N/A | N/A |
| N/A | N/A | qPCR | N/A | N/A | N/A | N/A |
| | | NTC | | | | |
| N/A | N/A | qPCR | N/A | N/A | N/A | N/A |
| | | NTC | | | | |

TABLE 6B
Summary of effect Cq and ΔCq for the different conditions
described in Example 4 on 2′-O-me-modified sRNA.

| | | | | ΔCq | ΔCq | ΔCq |
|---|---|---|---|---|---|---|
| | | siRNA | | (RNA − TE- | (2′OMe − | (low − high |
| Oligos added | Diluent | type | Cq | LPA) | unmod.) | [TSO]) |
| +siRNA +TSO | TE-LPA | siRNA | 19.1 | 0 | 0 | 0 |
| | Matrix 1:4 | siRNA | 19.71 | 0.61 | 0 | 0 |
| | Matrix 1:32 | siRNA | 19.45 | 0.35 | 0 | 0 |
| | TE-LPA | 2′OMe | 20.26 | 0 | 1.16 | 0 |
| | | siRNA | | | | |
| | Matrix 1:4 | 2′OMe | 21.84 | 1.58 | 2.13 | 0 |
| | | siRNA | | | | |
| | Matrix 1:32 | 2′OMe | 19.87 | −0.39 | 0.42 | 0 |
| | | siRNA | | | | |
| +siRNA +low | TE-LPA | siRNA | 21.15 | 0 | 0 | 2.05 |
| TSO | Matrix 1:4 | siRNA | 23.89 | 2.74 | 0 | 4.18 |
| | Matrix 1:32 | siRNA | 22 | 0.85 | 0 | 2.55 |
| | TE-LPA | 2′OMe | 22.74 | 0 | 1.59 | 2.48 |
| | | siRNA | | | | |
| | Matrix 1:4 | 2′OMe | 27.74 | 5 | 3.85 | 5.9 |
| | | siRNA | | | | |
| | Matrix 1:32 | 2′OMe | 23.3 | 0.56 | 1.3 | 3.43 |
| | | siRNA | | | | |
| +siRNA −TSO | TE-LPA | siRNA | N/A | N/A | N/A | N/A |
| | Matrix 1:4 | siRNA | N/A | N/A | N/A | N/A |
| | Matrix 1:32 | siRNA | N/A | N/A | N/A | N/A |
| | TE-LPA | 2′OMe | N/A | N/A | N/A | N/A |
| | | siRNA | | | | |
| | Matrix 1:4 | 2′OMe | N/A | N/A | N/A | N/A |
| | | siRNA | | | | |
| | Matrix 1:32 | 2′OMe | N/A | N/A | N/A | N/A |
| | | siRNA | | | | |
| −siRNA +TSO | TE-LPA | siRNA | N/A | N/A | N/A | N/A |
| | Matrix 1:4 | sRNA | N/A | N/A | N/A | N/A |
| | Matrix 1:32 | sRNA | N/A | N/A | N/A | N/A |
| | TE-LPA | 2′OMe | N/A | N/A | N/A | N/A |
| | | siRNA | | | | |
| | Matrix 1:4 | 2′OMe | N/A | N/A | N/A | N/A |
| | | siRNA | | | | |
| | Matrix 1:32 | 2′OMe | N/A | N/A | N/A | N/A |
| | | siRNA | | | | |
| N/A | N/A | qPCR | N/A | N/A | N/A | N/A |
| | | NTC | | | | |
| N/A | N/A | qPCR | N/A | N/A | N/A | N/A |
| | | NTC | | | | |

TABLE 6C
Summary of effect Cq and ΔCq for the different conditions
described in Example 4 on 2′-MOE-modified sRNA.

| | | | | ΔCq | ΔCq | ΔCq |
|---|---|---|---|---|---|---|
| | | siRNA | | (RNA − TE- | (2′MOE − | (low − high |
| Oligos added | Diluent | type | Cq | LPA) | unmod.) | [TSO]) |
| +siRNA +TSO | TE-LPA | siRNA | 18.44 | 0 | 0 | 0 |
| | Matrix 1:4 | siRNA | 19.14 | 0.7 | 0 | 0 |
| | Matrix 1:32 | siRNA | 18.58 | 0.14 | 0 | 0 |
| | TE-LPA | 2′MOE | 22.45 | 0 | 4.01 | 0 |
| | | siRNA | | | | |
| | Matrix 1:4 | 2′MOE | N/A | N/A | N/A | 0 |
| | | siRNA | | | | |
| | Matrix 1:32 | 2′MOE | 36.5 | 14.05 | 17.92 | 0 |
| | | siRNA | | | | |
| +siRNA +low | TE-LPA | siRNA | 20.22 | 0 | 0 | 1.78 |
| TSO | Matrix 1:4 | siRNA | 22.52 | 2.3 | 0 | 3.38 |
| | Matrix 1:32 | siRNA | 20.96 | 0.74 | 0 | 2.38 |
| | TE-LPA | 2′MOE | 25.35 | 0 | 5.13 | 2.9 |
| | | siRNA | | | | |
| | Matrix 1:4 | 2′MOE | N/A | N/A | N/A | N/A |
| | | siRNA | | | | |
| | Matrix 1:32 | 2′MOE | N/A | N/A | N/A | N/A |
| | | siRNA | | | | |
| +siRNA −TSO | TE-LPA | siRNA | N/A | N/A | N/A | N/A |
| | Matrix 1:4 | siRNA | N/A | N/A | N/A | N/A |
| | Matrix 1:32 | siRNA | N/A | N/A | N/A | N/A |
| | TE-LPA | 2′MOE | N/A | N/A | N/A | N/A |
| | | siRNA | | | | |
| | Matrix 1:4 | 2′MOE | N/A | N/A | N/A | N/A |
| | | siRNA | | | | |
| | Matrix 1:32 | 2′MOE | N/A | N/A | N/A | N/A |
| | | siRNA | | | | |
| −siRNA +TSO | TE-LPA | siRNA | N/A | N/A | N/A | N/A |
| | Matrix 1:4 | siRNA | N/A | N/A | N/A | N/A |
| | Matrix 1:32 | siRNA | N/A | N/A | N/A | N/A |
| | TE-LPA | 2′MOE | N/A | N/A | N/A | N/A |
| | | siRNA | | | | |
| | Matrix 1:4 | 2′MOE | N/A | N/A | N/A | N/A |
| | | siRNA | | | | |
| | Matrix 1:32 | 2′MOE | N/A | N/A | N/A | N/A |
| | | siRNA | | | | |
| N/A | N/A | qPCR | N/A | N/A | N/A | N/A |
| | | NTC | | | | |
| N/A | N/A | qPCR | N/A | N/A | N/A | N/A |
| | | NTC | | | | |

TABLE 6D
Summary of effect Cq and ΔCq for the different conditions
described in Example 4 on fully-modified sRNA.

| | | | | ΔCq | ΔCq | ΔCq |
|---|---|---|---|---|---|---|
| | | siRNA | | (RNA − TE- | (STC − | (low − high |
| Oligos added | Diluent | type | Cq | LPA) | unmod.) | [TSO]) |
| +siRNA +TSO | TE-LPA | siRNA | 18.79 | 0 | 0 | 0 |
| | Matrix 1:4 | siRNA | 19.49 | 0.7 | 0 | 0 |
| | Matrix 1:32 | siRNA | 18.9 | 0.11 | 0 | 0 |
| | TE-LPA | STC | 20.27 | 0 | 1.48 | 0 |
| | | siRNA | | | | |
| | Matrix 1:4 | STC | 22.82 | 2.55 | 3.33 | 0 |
| | | siRNA | | | | |
| | Matrix 1:32 | STC | 20.32 | 0.05 | 1.42 | 0 |
| | | siRNA | | | | |
| +siRNA +low | TE-LPA | siRNA | 21.04 | 0 | 0 | 2.25 |
| TSO | Matrix 1:4 | siRNA | 23.4 | 2.36 | 0 | 3.91 |
| | Matrix 1:32 | siRNA | 21.24 | 0.2 | 0 | 2.34 |
| | TE-LPA | STC | 20.16 | 0 | −0.88 | −0.11 |
| | | siRNA | | | | |
| | Matrix 1:4 | STC | 23.02 | 2.86 | −0.38 | 0.2 |
| | | siRNA | | | | |
| | Matrix 1:32 | STC | 20.47 | 0.31 | −0.77 | 0.15 |
| | | siRNA | | | | |
| +siRNA −TSO | TE-LPA | siRNA | N/A | N/A | N/A | N/A |
| | Matrix 1:4 | siRNA | N/A | N/A | N/A | N/A |
| | Matrix 1:32 | siRNA | N/A | N/A | N/A | N/A |
| | TE-LPA | STC | N/A | N/A | N/A | N/A |
| | | siRNA | | | | |
| | Matrix 1:4 | STC | N/A | N/A | N/A | N/A |
| | | siRNA | | | | |
| | Matrix 1:32 | STC | N/A | N/A | N/A | N/A |
| | | siRNA | | | | |
| −siRNA +TSO | TE-LPA | siRNA | N/A | N/A | N/A | N/A |
| | Matrix 1:4 | siRNA | N/A | N/A | N/A | N/A |
| | Matrix 1:32 | siRNA | N/A | N/A | N/A | N/A |
| | TE-LPA | STC | N/A | N/A | N/A | N/A |
| | | siRNA | | | | |
| | Matrix 1:4 | STC | N/A | N/A | N/A | N/A |
| | | siRNA | | | | |
| | Matrix 1:32 | STC | N/A | N/A | N/A | N/A |
| | | siRNA | | | | |
| N/A | N/A | qPCR | N/A | N/A | N/A | N/A |
| | | NTC | | | | |
| N/A | N/A | qPCR | N/A | N/A | N/A | N/A |
| | | NTC | | | | |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for processing a ribonucleic acid (RNA) molecule, comprising:
(a) providing a reaction mixture configured to produce a complementary deoxyribonucleic acid (cDNA) molecule from said RNA molecule, wherein said reaction mixture comprises:
(i) said RNA molecule;
(ii) a stem-loop primer that comprises:
a 5′ hemiprobe segment configured to hybridize to a first end region of said RNA molecule;
a stem-loop segment; and
a 3′ hemiprobe segment configured to hybridize to a second end region of said RNA molecule, and
(iii) a template switch oligonucleotide (TSO) that provides an exogenous template, wherein said TSO is configured for elongation of said RNA molecule when said RNA molecule is reverse-transcribed.

2. The method of claim 1, wherein said exogenous template comprises a sequence exogenous to said RNA molecule.

3. The method of claim 1, wherein said exogenous template comprises fewer than or equal to 10 consecutive bases identical to said RNA molecule.

4. The method of claim 1, wherein a complement of said exogenous template is not configured to bind to said RNA molecule.

5. The method of claim 1, wherein said RNA molecule is a naturally-occurring RNA molecule.

6. The method of claim 1, wherein said RNA molecule is obtained from a cell, a tissue, or an organism.

7. The method of claim 6, wherein said exogenous template comprises a sequence exogenous to said cell, said tissue, or said organism.

8. The method of claim 1, wherein said reaction mixture further comprises a cell or cellular RNA.

9. The method of claim 8, wherein said cell or cellular RNA comprises coding or noncoding RNA at least partially complementary to said RNA molecule.

10. The method of claim 1, further comprising producing said cDNA molecule from said RNA molecule.

11. The method of claim 1, wherein said stem-loop segment comprises a first synthetic primer hybridization sequence.

12. The method of claim 1, wherein said exogenous template of said TSO comprises a reverse complement of a second synthetic primer hybridization sequence.

13. The method of claim 12, further comprising detecting said cDNA molecule via said first synthetic primer hybridization sequence and said second synthetic primer hybridization sequence.

14. The method of claim 13, wherein said detecting further comprises quantitative PCR (qPCR), digital PCR (dPCR), sequencing, or array hybridization.

15. The method of claim 13, wherein said detecting further comprises contacting said cDNA molecule with a probe oligo configured to hybridize to an internal region of said cDNA molecule.

16. The method of claim 15, wherein said internal region of said cDNA molecule comprises a sequence complementary to said RNA molecule.

17. The method of claim 15, wherein said internal region of said cDNA molecule comprises a stem-loop segment within said cDNA molecule.

18. A method for processing an RNA molecule, comprising:
(a) providing in a reaction mixture under conditions suitable for extending said RNA molecule:
(i) said RNA molecule, which comprises a synthetic oligonucleotide or a synthetic linkage between two nucleotides of said RNA molecule; and
(ii) a stem-loop primer that comprises:
a 5′ hemiprobe segment configured to hybridize to a first end region of said RNA molecule;
a stem-loop segment comprising a first synthetic primer hybridization sequence; and
a 3′ hemiprobe segment configured to hybridize to a second end region of said RNA molecule; and
(b) extending said stem-loop primer using said RNA molecule as a template under conditions suitable to incorporate a 3′ region comprising a second synthetic primer hybridization sequence, thereby generating a cDNA molecule from said RNA molecule and said stem-loop primer.

19. A kit for detecting an RNA molecule, comprising:
(a) a stem-loop primer that comprises:
a 5′ hemiprobe segment configured to hybridize to a first end region of said RNA molecule;
a stem-loop segment; and
a 3′ hemiprobe segment configured to hybridize to a second end region of said RNA molecule; and
(b) a template switch oligonucleotide (TSO) providing an exogenous template, wherein said TSO is configured for elongation of said RNA molecule when said RNA molecule is reverse-transcribed.

20. A composition, comprising:
(a) an RNA molecule;
(b) a stem-loop primer that comprises:
a 5′ hemiprobe segment configured to hybridize to a first end region of said RNA molecule;
a stem-loop segment; and
a 3′ hemiprobe segment configured to hybridize to a second end region of said RNA molecule, and
(c) a template switch oligonucleotide (TSO) that provides an exogenous template, wherein said TSO is configured for elongation of said RNA molecule when said RNA molecule is reverse-transcribed.
